(19) United States
(12) Patent Application Publication
(10) Pub. No.: US 2023/0284929 A1
(43) Pub. Date: Sep. 14, 2023

(54) Vibrating Ingestible Capsule

(71) Applicant: Massachusetts Institute of Technology, Cambridge, MA (US)

(72) Inventors: Shriya Sruthi Srinivasan, Cambridge, MA (US); Carlo Giovanni TRAVERSO, Newton, MA (US); Robert S. LANGER, Newton, MA (US); Amro A. Alshareef, Cambridge, MA (US)

(73) Assignees: Massachusetts Institute of Technology, Cambridge, MA (US); The Brigham and Women's Hospital, Inc., Boston, MA (US)

(21) Appl. No.: 18/182,832

(22) Filed: Mar. 13, 2023

Related U.S. Application Data
(60) Provisional application No. 63/319,620, filed on Mar. 14, 2022.

(51) Int. Cl. A61B 5/07; A61H 23/02

(52) U.S. Cl. CPC A61B 5/073; A61H 23/0254

(57) ABSTRACT

The gastrointestinal capsule provides mechanical stimulation within the gastrointestinal tract. The capsule may be deployed in a subject's gastrointestinal tract orally by the subject ingesting the capsule. The capsule may mechanically stimulate any desired region within the gastrointestinal tract, including the stomach, small intestine, and large intestine. The mechanical stimulations provided by the capsule are applied to a portion of the inner walls or lining of a section of the gastrointestinal tract to simulate satiety.

### CROSS-REFERENCE TO RELATED APPLICATIONS

This application claims priority to U.S. Provisional Application No. 63/319,620 titled “VIBRATING INGESTIBLE CAPSULE,” filed Mar. 14, 2022, the entire disclosure of which is incorporated by reference.

### GOVERNMENT SUPPORT

This invention was made with government support under R01 EB000244 awarded by the National Institutes of Health. The government has certain rights in the invention.

### BACKGROUND

The treatment of complex diseases including obesity and diabetes continues to present healthcare challenges. The obesity epidemic, affecting nearly 45% of adults, increasingly engulfs healthcare resources by increasing the incidence of diabetes, hypertension, cancer, and heart disease. Given the inertia of behavioral modifications, cost, complications and invasiveness of bariatric surgery, and inadequacies of pharmacologic therapies, intragastric balloons (IGB) were developed as an alternative to treat obesity. Distension of the stomach by food contents triggers stretch mechanotransducers in the stomach to release satiety hormones and results in anorexigenic signaling. IGBs artificially trigger short-acting vagal afferents signaling stomach distension to induce early satiety.

Although providing short term weight loss, due to adaptation, IGBs failed to sustain changes in hunger or eating behavior after 10-12 weeks and failed to demonstrate superior efficacy to pharmacologic or surgical therapy. Neural adaptation to the chronic distension (as opposed to periodic distension during eating), and placement, removal, perforation, and obstruction complications pose challenges for the long-term efficacy and safety of IGBs. Following numerous deaths since 2016, the Food and Drug Administration (FDA) has issued several warning and some companies have recalled their IGB products.

Vagal nerve signaling plays a large role in satiation through a negative feedback loop of anorexigenic neurometabolic secretions in response to food intake largely based on volume, and not on the food's composition of carbohydrates, proteins, fats, or saline. This volume-dependent signaling is carried out by intraganglionic laminar endings (IGLEs), a prevalent type of vagal afferent innervating the gastric musculature, which sense contraction and distension and increase neuronal activity in the nucleus of the solitary tract where vagal afferents terminate and interact with reward, energy homeostasis, hunger, and mood circuitry. Vagotomies and electrical vagus nerve stimulation (VNS) have preclinically shown decreased weight and fat gain, food intake, and sweet cravings and increased satiation and energy expenditure. Clinically implemented for depression and epilepsy, VNS has shown a decrease in weight gain, reduced sweet cravings, and increased energy expenditure. However, non-GI side effects resulting from unspecific axonal targeting at the cervical level of the vagus and metabolic compensatory mechanisms have prevented clinical implementation in obesity. Furthermore, current VNS systems do not provide the complex signaling patterns physiologically present with food intake.

### SUMMARY

Embodiments of the present technology include an ingestible capsule. The ingestible capsule includes a housing forming a cavity, a vibrator, vibrating motor, or piezoelectric vibrating component disposed in the cavity, a power supply disposed in the cavity, and a biodegradable insulating membrane. The biodegradable insulating membrane is in electrical series with the vibrator and the power supply and in fluid communication with an exterior of the housing. The biodegradable insulating membrane is configured to dissolve in a fluid having a pH of 1.5 to 9, thereby closing a circuit connecting the power supply and the vibrator.

The vibrator in the ingestible capsule may include a motor with a shaft and a weight mechanically coupled to the shaft and radially offset from a longitudinal axis of the shaft. The shaft may be configured to rotate about the longitudinal axis of the shaft at a frequency of about 60 Hz to about 300 Hz. When the ingestible capsule is in a subject's stomach, the vibrator may be configured to rotate the weight about the longitudinal axis of the shaft to generate a centrifugal force, thereby stroking a portion of mucosa in the subject's stomach with the ingestible capsule.

The biodegradable insulating membrane may include glucose, gelatin, ellastolan, cellulose, or Eudragit. In one version, the biodegradable insulating membrane may be configured to dissolve in a fluid having a pH of 1.5 to 3.

The ingestible capsule may include a conductive or non-conductive spring that is in a compressed state when in contact with the biodegradable insulating membrane and in an expanded state after the biodegradable insulating membrane has dissolved. The ingestible capsule may include a pogo pin, where the spring is part of the pogo pin. The power supply may include an energy-harvesting mechanism, chemically charged power supply, wirelessly charged power supply, lithium-ion micro-battery, or silver oxide battery. A silver oxide battery, for example, may have a capacity of about 30 mAh to about 300 mAh.

The ingestible capsule's cavity may be a first cavity and the housing may include a first section, a second section press-fittingly coupled with the first section to form the first cavity, and a third section press-fittingly coupled with the second section to form a second cavity. The second cavity may contain the biodegradable insulating membrane. The third section may have a conduit for fluid communication between the second cavity and the exterior of the housing.

The housing may include a protruding member disposed on an outer surface of the housing. The protruding member may have a helical or grooved shape. The protruding member may include a plurality of studs protruding from an outer surface of the housing.

When the ingestible capsule is in a stomach of a subject who has ingested the ingestible capsule, closing the circuit connecting the power supply and the vibrator can cause the vibrator to vibrate the ingestible capsule at a frequency that induces a feeling of satiety in the subject, an illusory insufflation of the stomach, and/or serotonin release in the subject and/or causes the ingestible capsule to stimulate mucosal receptors in the stomach.

Another embodiment of the present technology includes an ingestible capsule. The ingestible capsule includes a housing forming a cavity, an actuator disposed in the cavity, and a power supply disposed in the cavity. The actuator is configured to oscillate about a longitudinal axis of the ingestible capsule at a frequency of about 60 Hz to about 120 Hz, thereby causing the ingestible capsule to rotate. The power supply is configured to provide power to the actuator.

When the ingestible capsule is in a subject's stomach, rotation of the ingestible capsule may cause the ingestible capsule to stroke a portion of mucosa in the subject's stomach.

Another embodiment of the present technology includes a method of stimulating a sensation of satiety in a subject. The method includes, with an ingestible capsule, stroking a portion of mucosa in the stomach at a frequency of about 60 Hz to about 120 Hz to induce an illusory insufflation of the stomach.

The method may also include closing a circuit connecting a power supply and a vibrator in the ingestible capsule to induce the stroking by dissolving, with stomach fluid, water, or an ingested liquid, a biodegradable insulating membrane disposed in electrical series between the power supply and the vibrator. The stroking the portion of mucosa may include radially oscillating the ingestible capsule about a longitudinal axis of the capsule with the vibrator. The method may also include, while stroking the portion of mucosa, stimulating mucosal receptors in the stomach, inducing cephalic phase activity, and mimicking food intake with the ingestible capsule to induce serotonin release in the subject. The method may also include orally ingesting the ingestible capsule.

Another embodiment of the present technology includes an ingestible capsule. The ingestible capsule includes a housing forming a cavity, a vibrator disposed in the cavity, a power supply disposed in the cavity, a biodegradable insulating membrane, and a conductive spring. The biodegradable insulating membrane is disposed in electrical series with the vibrator and the power supply and in fluid communication with an exterior of the housing. The biodegradable insulating membrane is configured to dissolve in stomach acid. The conductive spring is held in a compressed state when in contact with the biodegradable insulating membrane and an expanded state closing a circuit between the vibrator and the power supply after the biodegradable insulating membrane is dissolved, thereby causing the vibrator to move about a longitudinal axis of the ingestible capsule at a frequency of about 60 Hz to about 120 Hz. The motion of the vibrator causes the ingestible capsule to stroke a portion of mucosa when the ingestible capsule is in a subject's stomach.

The outer surface of the ingestible capsule may be textured with at least one of a protrusion or depression, for example, a helical depression. The at least one protrusion or depression may include a plurality of protruding studs disposed in the helical depression. Each protruding stud in the plurality of protruding studs may have a diameter of about 200 μm to about 800 μm. The at least one protrusion or depression may include a plurality of slits, which may be uniform or varying in size and/or shape.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1A is a schematic illustration of a vibrating ingestible capsule.

FIG. 1B is a schematic illustration depicting an actuation mechanism for an ingestible capsule.

FIG. 2 is another schematic illustration of an ingestible capsule.

FIG. 3 is a photograph of an ingestible capsule.

FIG. 4A is a force diagram of an ingestible pill with a rotating motor weight.

FIG. 4B is another view of the force diagram in FIG. 4A.

FIG. 5 shows steps to assemble an ingestible capsule.

FIG. 6A is a schematic illustration of an ingestible capsule having a helical pattern on the outer surface of the capsule.

FIG. 6B shows a photograph of the capsule in FIG. 6A.

FIG. 7A is a schematic illustration of an ingestible capsule having a studded pattern on the outer surface of the capsule.

FIG. 7B shows a photograph of the capsule in FIG. 7A.

FIG. 8A is a schematic illustration of a cross-section of a helical pattern on the outer surface of an ingestible capsule.

FIG. 8B is a schematic illustration of the ingestible capsule having the helical pattern in FIG. 8A.

FIG. 9A is a schematic illustration of a cross-section of another helical pattern on the outer surface of an ingestible capsule.

FIG. 9B is a schematic illustration of the cross-section of an ingestible capsule having the helical pattern in FIG. 9A.

FIG. 9C is a schematic illustration of the ingestible capsule having the helical pattern in FIG. 9A.

FIG. 10A is a schematic illustration of a cross-section of another helical pattern on the outer surface of an ingestible capsule.

FIG. 10B is a schematic illustration of the cross-section of an ingestible capsule having the helical pattern in FIG. 10A.

FIG. 10C is a schematic illustration of the ingestible capsule having the helical pattern in FIG. 10A.

FIGS. 11A-11C illustrate a Vibrating Ingestible BioElectronic Stimulator (VIBES).

FIG. 11A shows how the VIBES pill contacts the gastric lining and activates following contact with gastric fluid. Vibrations activate IGLEs in the celiac plexus, signaling distension to the NTS, which interacts with hunger circuitry to signal illusory distension.

FIG. 11B shows how the VIBES pill sits amongst gastric rugae in a swine stomach and strokes the mucosa as it performs stimulation.

FIG. 11C shows that the VIBES pill includes 1) an offset motor, 2) silver oxide battery, 3) central body, 4) motor cap, 5) pill cap, 6) pogo pin, 7) gelatinous membrane, and 8) resistor (e.g., with a resistance of 0-120 ohms).

FIGS. 12A-12G show gastric afferent electrophysiology of stretch-sensitive mechanoreceptors.

FIG. 12A shows insufflation of the gastric cavity performed to 30%, 60%, and 90% of the gastric volume while recording electroneurography (ENG) from celiac vagal branches demonstrating spiking in response to stretch. VIBES at 60, 80, and 100 Hz resulted in similar spiking behavior (black). Rectified ENG signals are plotted.

FIG. 12B shows a raw ENG signal from a stretch sensitive afferent fiber responding to mechanical inflation.

FIG. 12C shows neural activity produced by a VIBES pill within the stomach.

FIG. 12D shows neural activity produced by a VIBES pill after bilateral vagotomy.

FIG. 12E shows periodic VIBES vibration resulted in repeatable induction of the stretch response.

FIG. 12F shows the afferent response to mucosal stroking of the gastric lumen by an endoscopic fiber.

FIG. 12G shows the VIBES pill rotation against the mucosal surface.

FIG. 13 shows VIBES neuromodulation of gastric vagal afferents yields illusory metabolic satiety. An experimental schematic for blood sampling during VIBES stimulation between 30-60 minutes and the response of hormones normalized to their baseline levels in animals with no stimulation (lower traces) and VIBES (upper traces) are shown.

FIGS. 14A-14J show the VIBES effect on feeding and weight gain.

FIG. 14A shows the percentage of meal consumed by swine in the VIBES, PEG-control, and control groups.

FIG. 14B shows the percentage of the meal consumed by animals A, B, C, and D over two weeks treated on VIBES and two weeks with no treatment (control).

FIG. 14C shows the energy consumed at each meal (dots) by animals A, B, C, and D over two weeks treated on VIBES, PEG-control or no treatment (control). Bars represent median and quartiles.

FIG. 14D shows the consumption of animals A (squares), B (triangles), and C (diamonds) in a cross-over study design comprising 3 meals with VIBES treatment, no treatment and VIBES, consecutively.

FIG. 14E shows the weight gain rate for each animal when treated with VIBES and no treatment.

FIG. 14F shows the duration of time that animals spent in each category of behavior.

FIG. 14G shows the percentage of time spent in each behavior for the control group.

FIG. 14H shows the percentage of time spent in each behavior for the VIBES group.

FIG. 14I shows the probability of active behavior at each hour.

FIG. 14J shows the probability of feeding behavior at each hour.

FIG. 15 shows a chemical resistance test. Submersion in simulated gastric fluid did not erode the pills surface or damage any internal hardware (bottom) as compared to its pre-submersion state (top). Following 24 hours of submersion, the pill was able to be activated and functioned normally.

FIG. 16 shows thermal testing. The VIBES was operated at various frequencies for 30 minutes in 20 mL of saline. Change in temperature from baseline was assessed using thermal imaging of the fluid. In all cases, there was less than a 0.5° C. increase in the surrounding fluid, indicating that the VIBES does not pose any thermal risk to tissue.

FIG. 17 shows the insufflation of the stomach to 30% (left) and 90% (right) can be visualized by the presence of lack of rugae in the stomach (white arrow). Circled is the VIBES pill making mucosal contact.

FIG. 18 shows the afferent ENG in response to VIBES stimulation at frequencies between 24 Hz and 500 Hz.

FIG. 19 shows the rectified electroneurography demonstrates a sharp increase in spiking 0-12 seconds following the beginning of inflation. In this representative trial, the gastric cavity was insufflated to 90% of its full volume, within 38 seconds of insufflation.

FIG. 20 shows the serotonin release on the luminal surface of gastric tissue (n=12 trials using tissue samples from n=2 swine) was assayed in response to VIBES at varying frequencies.

FIGS. 21A-21D show different surface geometries for mucosal stroking.

FIG. 21A shows the surface features of studs that were incorporated to stroke microvilli and rugae during VIBES pill rotation.

FIG. 21B shows the surface features of spirals that were incorporated to stroke microvilli and rugae during VIBES pill rotation.

FIG. 21C shows the effect of surface geometry on serotonin release indicates significantly higher serotonin release levels (p<0.05, Student's two tailed t-test) for a spiral design as compared to a straight surface geometry or the control condition, where no VIBES was used.

FIG. 21D shows a representative image of the spiral VIBES pill seated amongst gastric ruggae.

FIG. 22 shows representative tissue cross sections stained with hematoxylin and eosin from control untreated animals (left side) and animals treated with the VIBES pill (right side). Tissue treated with VIBES shows no marked inflammation, irritation, or morphological changes.

FIG. 23 shows a radiographic image of the VIBES pill and barium tracking pellets. The VIBES pill (middle circle) and barium pellets (upper and lower circles) can be seen in the GI tract one day following oral administration in a swine in this radiographic image.

FIG. 24 shows the composition and activation mechanism of the VIBES pill.

FIG. 25 shows the tethered VIBES pill for placement and maintenance through a PEG tube.

FIG. 26 shows the behavioral model, adapted from Lehner's model.

FIGS. 27A-27D show the behavioral model analysis pipeline.

FIG. 27A shows the data collection. The data was collected between September 2021 and February 2022. The data was collected 24/7 across two pens (one camera each). There were 1-hour segments of continuous video and audio at 1080p HD, night vision, 80-degree FOV. The audio was 16000 Hz, mono, fltp, 15 kb/s. Each file is approximately 400 Mb. The data was stored in AWS MIT Cloud computing Servers (E2). High throughput, low latency high performance computing clusters. NVIDIA A100 Tensor Core GPUs.

FIG. 27B shows the markerless pose estimation. The training data for markerless pose estimation model was >400 labeled images of body parts and objects in the environment from 2 camera perspectives. The range of color data was from red-green-blue (RGB) to black and white (B/W). The data output was positional coordinate time-series data. The coordinates of 22 body parts (e.g., snout) and 10 objects (e.g., gear toy).

FIG. 27C shows the conditional random fields and tracking trajectories. The data was manipulated from the positional coordinate time-series data of body parts, the relative location at each time step was calculated. The training data was time-series data of the relative location of the head, snout, and tail (window=39). The data output estimated behaviors being conducted at time interval t.

FIG. 27D shows the behavior characterization. The plot of the estimated behaviors investigate inter- and intra-individual trends.

FIG. 28 shows the markerless pose estimation: the generated features provide an estimate of body angle and distance.

FIG. 29 shows the results of a DeepLabCut prediction model.

FIG. 30 shows the confusion matrix of results for ethogram prediction/accuracy across time.

### DETAILED DESCRIPTION

The gastrointestinal capsules (also referred to herein as the capsules, ingestible capsules, or pills) disclosed here provide mechanical and neural stimulation within the gastrointestinal tract. In one example, a capsule may be deployed in a subject's gastrointestinal tract orally by the subject ingesting the capsule. In another example, a capsule may be deployed in the gastrointestinal tract by inserting the capsule into the gastrointestinal tract via endoscope or colonoscope. A capsule may also be placed into the stomach via a percutaneous gastrostomy tube (PEG tube). A capsule may mechanically stimulate any desired region within the gastrointestinal tract, including, for example, the stomach, small intestine, sphincters and/or large intestine. An exemplary capsule sinks through gastric or other luminal contents and sustains contact with the gastrointestinal lining because of its total weight and density (e.g., a density greater than 2 g cm⁻³). Preferably, the mechanical stimulations provided by the capsule are applied to a portion of the inner walls or lining of a section of the gastrointestinal tract. In one embodiment, the capsule mechanically stimulates a portion of stomach mucosa or stomach lining with sufficient amplitude to stimulate mucosal tissue and/or mechanoreceptors in the mucosal, submucosal, muscularis, and/or serosal layers. An exemplary capsule is naturally evacuated with the stool without obstruction, perforation, or distress. An exemplary capsule may only include low-cost components, so it does not need to be reacquired post evacuation. Similarly, an exemplary capsule need not be recharged.

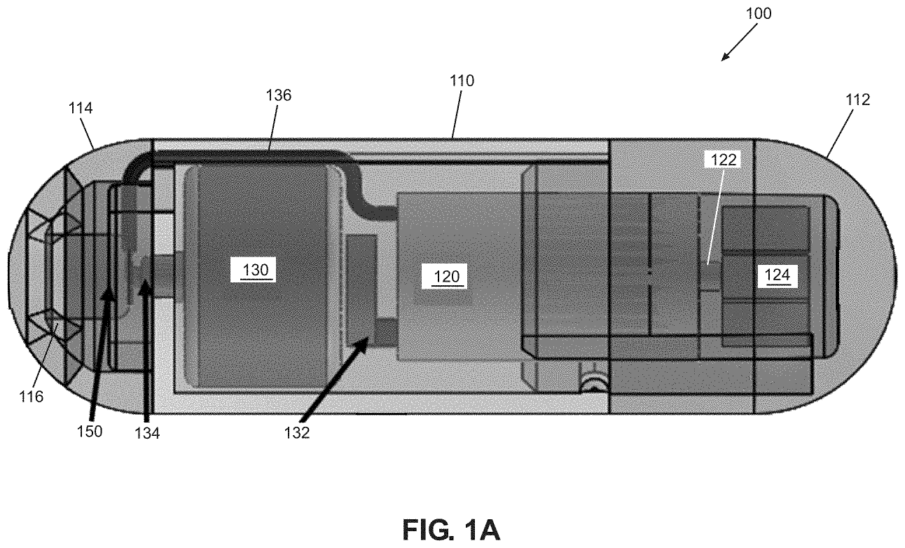

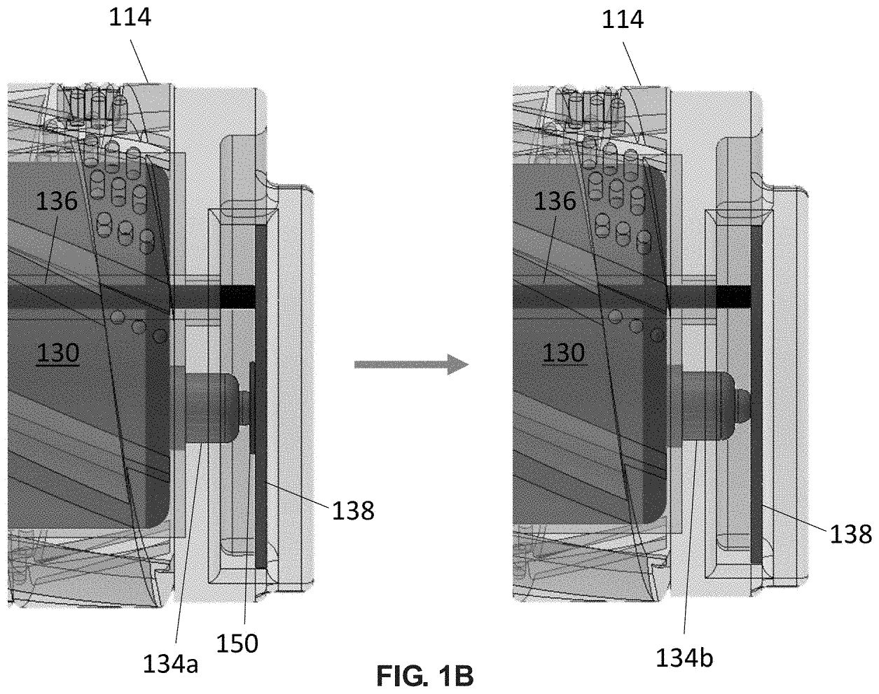

FIG. 1A shows a gastrointestinal capsule 100 that provides mechanical stimulation within the gastrointestinal tract. The gastrointestinal capsule 100 includes a capsule housing including a middle housing 110 and end caps 112 and 114 coupled to opposite ends of the middle housing 110. The capsule housing 110 creates a sealed main cavity in which electronic components are protected from any fluids that the capsule 100 encounters. Inside the main cavity are a motor 120 and a battery 130 or other power supply, such as an energy-harvesting mechanism or wirelessly or chemically charged power supply, configured to provide power to the motor 120. The motor 120 rotates a shaft 122 mechanically coupled to the motor 120. A weight 124 attached to the shaft in a position so that its center of mass is centered on or laterally offset from the central longitudinal axis of the shaft. The distance between the weight 124 and the motor 120 is limited so as to reduce the total volume of the capsule, but long enough so that the motor body does not interfere with the rotation of the weight. As an example, the weight 124 may have a semi-circular shape with a radius of 2.5 mm. The battery 130 is electrically coupled to the motor 120 in electrical series. A resistor 132 may be electrically coupled in series with the motor 120 and the battery 130 to drop the voltage supplied from the battery 130 to the motor 120. A spring 134 (e.g., a pogo pin, a compression spring, a spring clip, or other spring-loaded connector), which be conductive or non-conductive, and one or more conductive connectors 136 including wires (e.g., rubber coated copper wires) or cables may also be part of the electrical circuit in the capsule 100.

When the motor 120 is operated, the shaft 122 rotates, causing the weight 124 to also rotate. The motor may be a miniature coreless motor. Coreless motors are preferable because of their high efficiency, high acceleration rates, low inertia, and high power to size ratio. The movement of the weight 124 within the capsule causes capsule movement. Depending on the frequency of rotation, the size of the weight, the type of media that the capsule is in, and the type of surface that the capsule is disposed on, the capsule may move in one or more different ways, including rocking, sweeping, rotating, oscillating, vibrating, and/or teeter-tottering. For example, when the capsule 100 is in contact with a plical surface, it will rotate. If the capsule 100 is unconstrained on its sides, it will rock back and forth. The pattern of capsule movement also changes when the capsule 100 is in contact with bumps and/or grooves in the tissue. These capsule movements provide mechanical stimulation to a portion of tissue within the gastrointestinal tract.

The placement of the weight within the capsule also determines the type of capsule movement. For example, the center of mass of the weight may be centered on or laterally offset and/or longitudinally offset from the center of the capsule. In a version, the center of mass of the weight is laterally offset from the central longitudinal axis of the capsule by about 1 mm to about 2 mm. In the same or a different version, the center of mass of the weight is centered on the lateral axis or longitudinally offset from the center of the capsule up to the edge of the capsule's end cap (e.g., 1 mm, 2 mm, 3 mm, 4 mm, 5 mm, 6 mm, 7 mm, 8 mm, 9 mm, 10 mm, 11 mm, 12 mm, or 13 mm from the center of the capsule). In a version, the center of mass of the weight is longitudinally offset from the center of the capsule by about 11.2 mm. When the center of mass of the weight is longitudinally offset, rotation of the weight about the motor's shaft creates a teeter-totter motion in the capsule. The distance of longitudinal offset determines the amplitude of the teeter-totter motion. In a version, one end cap of the capsule is mechanically coupled to the motor shaft and the other end cap is mechanically coupled to the motor body so that both end caps rotate relative to each other at a rate proportional to their respective masses, so that the two sides of the capsule rotate in opposite directions to facilitate mixing.

In some embodiments of the capsule, the outer casing of the motor itself can serve as the outer shell, or a portion of the outer shell, of the capsule. This would allow for a smaller size capsule by eliminating an additional layer over the motor. Having the outer casing of the motor serves as at least a portion of the capsule's outer shell may also facilitate relative rotation of the capsule's end caps.

The weight rotation frequency may be about 2 Hz to about 400 Hz (e.g., 2 Hz, 5 Hz, 10 Hz, 20 Hz, 30 Hz, 40 Hz, 50 Hz, 60 Hz, 70 Hz, 80 Hz, 100 Hz, 120 Hz, 150 Hz, 180 Hz, 200 Hz, 250 Hz, 300 Hz, 350 Hz, or 400 Hz). Preferably, the frequency is about 60 Hz to about 120 Hz. Operation of the motor at these frequencies over the operational time period does not generate enough heat to cause any tissue damage. Operation of the motor at these frequencies also does not cause abrasion, irritation, or inflammation of the tissue. As an example, operation of the motor may cause capsule displacement amplitudes of about 0 mm to about 5 mm when powered with a 1.55 V silver oxide battery.

FIG. 1B shows an actuation assembly in one embodiment of the capsule 100. In this embodiment, the circuit is completed and the motor 120 begins receiving power when a membrane 150 (or a thin layer, barrier, coating, film, or sheet) is dissolved or degraded. The left image in FIG. 1B shows the actuation assembly in the pre-actuation state where the motor 120 is not receiving power. The right image in FIG. 1B shows the actuation assembly in the actuated state where the electrical circuit is completed and the motor 120 is receiving power. In the left image in FIG. 1B, the spring in the pogo pin 134a is compressed and a surface of the pogo pin's plunger is in direct contact with a surface of the membrane 150. The membrane 150 is disposed between the pogo pin's plunger and a mating receptacle 138 (e.g., a target or a land, having a flat or concave conductive surface) for the pogo pin 134b to engage, to complete the connection path when the membrane 150 is no longer present. When the membrane 150 degrades or dissolves, the pogo pin plunger extends under the force of the pogo pin's spring to contact the mating receptacle 138. A conductor 136 electrically connects the mating receptacle 138 to the battery 130. If desired, a capacitor (not shown) can form an RC delay element with the resistor 132 that introduces a time delay to offset the activation of the motor 120 in response to the conductor 136 contacting the mating receptacle 138.

The membrane 150 degrades or dissolves over a desired period of time when in contact with fluid in a particular pH range in order to complete the circuit to the motor 120. For example, the desired period of time may be about 1 minute to about 2 hours (e.g., 1 minute, 2 minutes, 3 minutes, 4 minutes, 5 minutes, 6 minutes, 7 minutes, 8 minutes, 9 minutes, 10 minutes, 20 minutes, 30 minutes, 1 hour, or 2 hours). In one version, the membrane 150 dissolves in about 5 minutes (e.g., 4.3±1.2 minutes), soon after it contacts the fluid in the set pH range. The set pH range may be about 2 to about 9, or any sub-range therein. In one version, the pH range is about 1.5 to about 3.5, so that the membrane 150 degrades or dissolves, for example, when it contacts gastric fluid. The membrane 150 may be insoluble or have a sufficiently slow dissolution/degradation rate in fluids outside the set pH range to prevent capsule activation when the capsule is in a fluid outside the set pH range. The thickness of the membrane 150 may be selected so that it dissolves or degrades in a desired amount of time. The membrane 150 may have a thickness of about 0.5 mm to about 5 mm. Preferably, the membrane 150 has a thickness of about 0.5 mm to about 2.5 mm. More preferably, the membrane 150 has a thickness of about 0.5 mm to about 1 mm.

The membrane 150 may be a polymer with pH sensitive chemical bonds that are cleaved in a particular pH range. For example, the cleavable bonds may include imine bonds, hydrozone bonds, oxime bonds, amide bonds, acetal bonds, orthoester bonds, acrylate bonds, and/or methacrylate bonds. The membrane 150 may include glucose, gelatin, chitosan, ellastolan, cellulose, Eudragit, poly lactic-co-glycolic acid (PLGA), polylactic acid (PLA), polycarbonate (PC), polycarboxylic acid (PCA), polyglycolide (PGA), and/or polymethacrylate. Preferably, the membrane 150 includes Eudragit. The membrane 150 may be a biocompatible material so that when it degrades or dissolves inside the body, it is not harmful to living tissue.

In one example, the membrane 150 may be a discrete shape (e.g., circle or rectangle) that is as small as the diameter of the pogo pin's plunger or as big as the capsule 100 itself. In another example, the membrane 150 may be part of a layer or coating disposed on the exterior surface of the capsule's housing.

In one example, the membrane 150 may be disposed in a cavity formed in the end cap 114. This end cap cavity is in fluid communication with fluid outside of the capsule 100. For example, the end cap 114 may have one or more openings or conduits 160 in the end cap 114 so that fluid can move between the interior of the end cap cavity and the exterior of the capsule 100. The main cavity is sealed off from the end cap cavity with medical-grade adhesive or sealant so that fluid does not enter the main cavity. At least part of the pogo pin 134 plunger and the mating receptacle 138 are disposed in the end cap cavity. In another version, the membrane 150 and portions of the pogo pin 134 and the mating receptacle 138 are disposed on an exterior surface of the capsule 100 (e.g., the exterior of the central housing), where they freely interact with fluid in the environment of the capsule 100. For example, the seal may be formed by filling the connection points and wire/pogo pin through-holes in the main cavity with a seal that is impermeable to GI fluids (e.g., medical grade epoxy). The pogo pin 134 plunger and the mating receptacle 138 may be made of one or more biocompatible conductive materials, including gold, platinum, or palladium. The pogo pin 134 and the mating receptacle may also be chemically resistant to gastrointestinal fluids.

Alternatively, the capsule 100 may include one or more sensors (not shown), such as an accelerometer, temperature sensor, pH sensor, or piezoelectric sensors, instead of a dissolvable membrane. When the sensor senses when the capsule 100 has entered the desired region of the gastrointestinal tract, it triggers the motor 120. The capsule 100 could also include a wireless receiver or antenna that receives a wireless signal, such as a Bluetooth low energy (BLE) signal, from a device outside the body and triggers the motor 120 in response to the signal.

The battery 130 may be a primary battery that provides power to the motor for up to about 2 hours. For example, the battery 130 may power the motor for 10 minutes, 20 minutes, 30 minutes, 40 minutes, 50 minutes, 1 hour, 1.5 hours, or 2 hours. Preferably, the battery 130 powers the motor for about 30 minutes to about 40 minutes. The battery 130 may be a silver oxide battery, a lithium battery, a copper-zinc battery, or a zinc-carbon battery. The battery may supply a voltage of 1.55 volt (V) to about 3 V. For example, the battery may supply a voltage of 1.55 V, 1.60 V, 1.65 V, 1.7 V, 1.8 V, 1.9 V, 2.0 V, 2.2 V, 2.5 V, 2.8 V, or 3.0 V. Preferably, the battery 130 is a silver oxide battery, which is very biocompatible and used in several FDA-approved devices, with a voltage of 1.55 V. The battery may have a capacity of 30 milliamp-hours (mAh) to about 300 mAh (e.g., 30 mAh, 50 mAh, 80 mAh, 100 mAh, 150 mAh, 200 mAh, 250 mAh, or 300 mAh), and the capacity may be chosen depending on the desired operation time of the motor 120 and the size of the pill (capsule). In one example, the silver oxide battery has a capacity of 80 mAh and operates for about 30 minutes to about 40 minutes. The electrical circuit may include a resistor 132 to drop the voltage supplied to the motor 120 in order to control the motor's frequency. The resistor may have a resistance between about 0 ohms and about 10,000 ohms (e.g., 120 ohms).

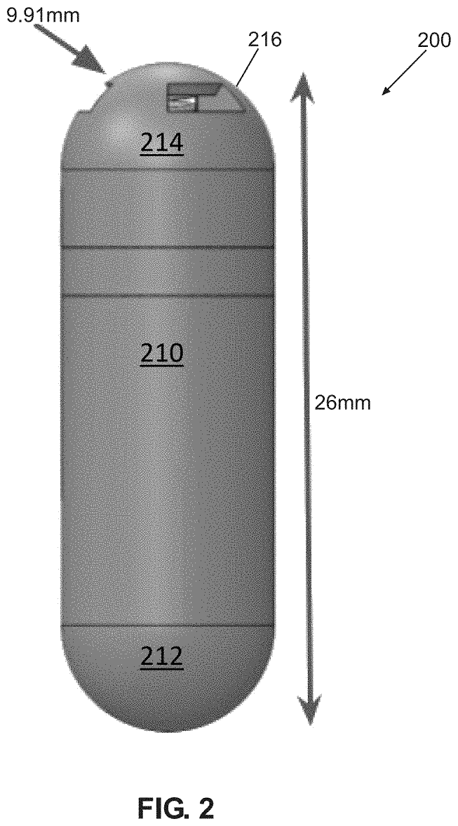

FIG. 2 shows the outer housing of a gastrointestinal capsule 200. The capsule 200 includes middle housing 210 and end caps 212 and 214 coupled to opposite ends of the middle housing 210. The capsule size may be zero (0), double zero (00), or triple zero (000). In one example, the capsule 200 is a triple-zero (000) capsule, with a length of about 26 mm and a diameter of about 9.91 mm. The housing components are rigid, biocompatible, and chemically stable within the environment of the gastrointestinal tract. In some versions, the housing may also be transparent. As an example, the housing material may be VeroClear, a photopolymer that simulates polymethylmethacrylate (PMMA), PMMA, gelatin, hydroxypropyl cellulose, ellastolan, and/or pullulan. The thickness of the housing walls is chosen to provide enough space for the electronic components while still being manufacturable and rigid enough to transmit vibrational force. For example, the thickness may be about 0.4 mm to about 1 mm (e.g., 0.4 mm, 0.5 mm, 0.6 mm, 0.7 mm, 0.8 mm, or 1.0 mm). Preferably, the thickness of the housing wall is about 0.6 mm. The capsule 200 includes a conduit 216 though which fluid may flow to dissolve the membrane in the end cap cavity to activate the capsule's motor. The three housing sections 210, 214, and 216 may be press-fit together to create a tightly sealed main cavity. The three housing sections 210, 214, and 216 may also be sealed together and/or coated with biocompatible adhesive to maintain a fluid-proof seal. In other embodiments, the housing may be a single piece or two pieces instead of three. The housing components may be 3D printed or injection molded (e.g., via two-shot molding or overmolding).

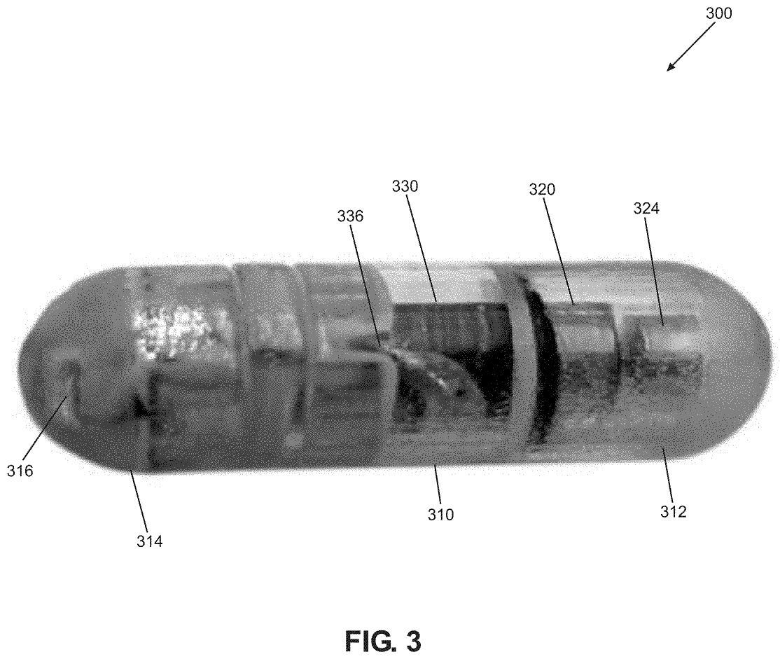

FIG. 3 is a picture of a gastrointestinal capsule 300. The capsule 300 includes a capsule housing including a middle housing 310 and end caps 312 and 314 coupled to opposite ends of the middle housing 310. The capsule housing creates a sealed main cavity in which electronic components are protected from any fluids that the capsule 300 encounters. Inside the main cavity are a motor 320 and a battery 330 configured to provide power to the motor 320. The motor 320 rotates a shaft mechanically coupled to the motor 320. A weight 324 is attached to the shaft in a position so that its center of mass is laterally offset from the central longitudinal axis of the shaft. A conduit 316 in the end cap 314 provides fluid coupling between a separate cavity in the end cap 314 and the external environment of the capsule 300. The capsule 300 includes an actuation assembly that includes a membrane disposed between a conductive spring and a conductive connector 336. The membrane dissolves or degrades when in contact with a fluid of a set pH, thereby closing the electrical circuit and actuating motor operation. The membrane can also be tuned to dissolve to a temperature cue.

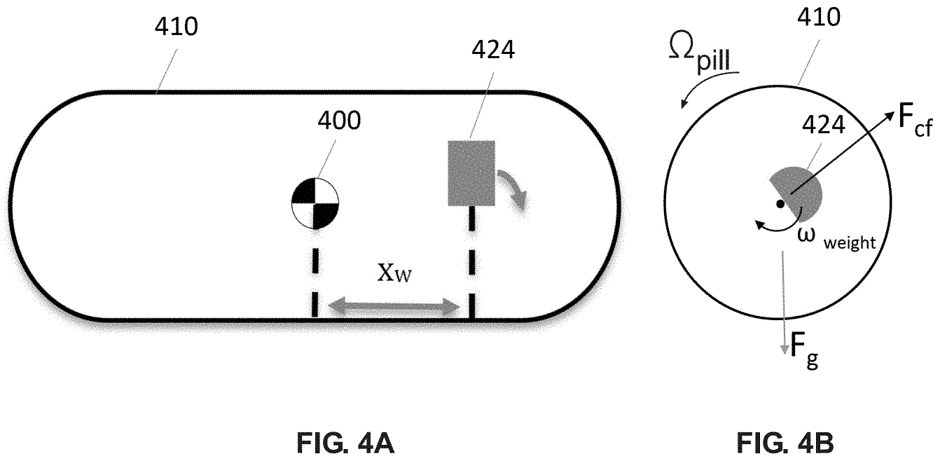

FIGS. 4A and 4B show two different views of a force diagram of an ingestible capsule with a rotating weight 424 offset from the center point of the capsule 400 inside the capsule's housing 410. Rotation of the weight 424 generates a centrifugal force, F_{cf}, causing the capsule housing 410 to move against surface friction. F_{cf} pulls the capsule housing 410 radially outward and changes the capsule housing's direction with the offset weight 424:

$${F}_{cf} = {m}_{weight} * {ω}_{weight}^{2} * {r}_{weight} \qquad ( 1 )$$

$$f = \frac{{ω}_{weight}}{2 * pi} \qquad ( 2 )$$

where ω_{weight} is the angular velocity of the weight 424 and r_{weight} is the radial offset of the weight 424 from the central point 400 of the capsule. The resulting vibrational frequency of the capsule is f, The oscillatory movement of the capsule is caused by the offset of this force to one side of the capsule by x_{w} from the center of mass 400, which causes the capsule to rock as the weight 424 moves with and against the force of gravity.

ΣL=L_{weight}L_{capsule}=0 (3)

I_{weight}* ω_{weight}=I_{capsule}* Ω_{capsule}given ω_{weight}>>Ω_{capsule} (4)

The rotational movement of the capsule is governed by conservation of momentum within the system. In a frictionless environment, as the motor within the capsule spins at ω_{weight}, the capsule counters that spin with an angular velocity Ω_{capsule}, proportional to the rotational rate of the motor and scaled by a ratio of the moment of inertia of the weight, I_{weight}, to that of the capsule.

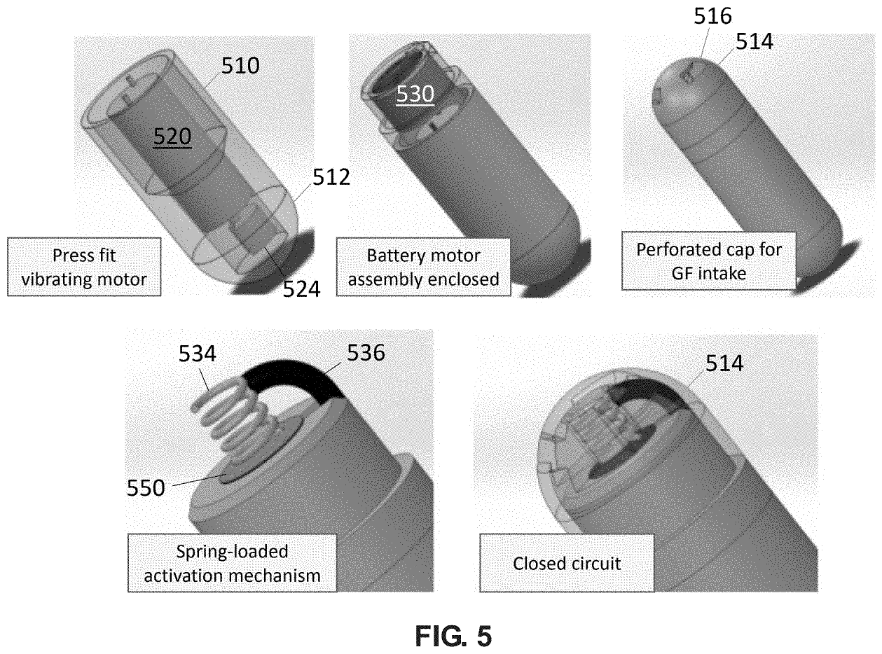

FIG. 5 shows a method of assembling a gastrointestinal capsule. The capsule is split into three sections: the motor cap (or end cap) 512, the central body (or middle housing) 510, and the pill cap (or end cap) 514. The vibrating motor 520, mechanically coupled to an offset weight 524, is first pressed into the motor cap 512. The battery 530 is then placed in the central body 510. A copper pad is soldered on the positive lead of the motor 520 and positioned near the positive lead of the battery 530. The spring-loaded pogo pin 534 is then pressed into the central body 510, so that the battery's negative terminal is in contact with the pogo pin 534. The central body 510 assembly is then pressed onto the motor cap 512 assembly such that the positive terminal of the battery 530 contacts the positive copper pad on the motor 520. The negative lead of the motor 520 is stretched through the central body 510 to the pill cap 514. The negative lead is soldered onto a conductive pad (or mating pad) in the top section that when closed onto the pill, closes the circuit by contacting the pogo pin 534. A membrane 550 is also placed between the negative lead pad and the pogo pin 534. The membrane 550 dissolves once the pill reaches the desired gastrointestinal fluid, so that the capsule is activated only when it reaches the desired section of the gastrointestinal tract.

The outer surface of the capsule housing may be smooth or may be microtextured. The surface geometry of the outer surface may be selected for a particular application. For example, microtexturing the surface may increase or reduce drag, and/or increase or reduce capsule rotation. Microtexturing may promote smooth rotations of the capsule about the longitudinal axis. Alternatively, microtexturing may promote capsule rocking movements or vibrations that help the capsule push down into the mucosa. Microtexturing can stimulate mechanoreceptors and induce stroking motions on the mucosa. Microtexturing can also clear mucosal contents, wick away gastric fluid, and interact with food particles.

Microtexturing may include helical patterns (also called spiral patterns), stud patterns (also called nub, bump, or nodule patterns), or slit patterns, which may be uniform or variable in size and/or shape. The microtexturing may protrude out from the surface of the housing or may intrude into the surface of the housing (e.g., as a groove). The microtexturing may be disposed over the entire outer surface of the housing or on only a portion (e.g., only on the central part of the housing or only on the end caps). More than one type of microtexturing may be included (e.g., both stud and helical patterns). The microtexturing may be formed into the capsule housing or may be a separate layer that is disposed onto the capsule housing. The microtextured housing or microtextured layer may be formed by 3D printing, injection molding, laser cutting, laser grooving, press molding, extrusion, thermoforming, texturing using mills, texturing using abrasive materials, texturing using molding, texturing using polymer casting, and/or blow molding. One-shot or multi-shot molding may be used to form microtextured components.

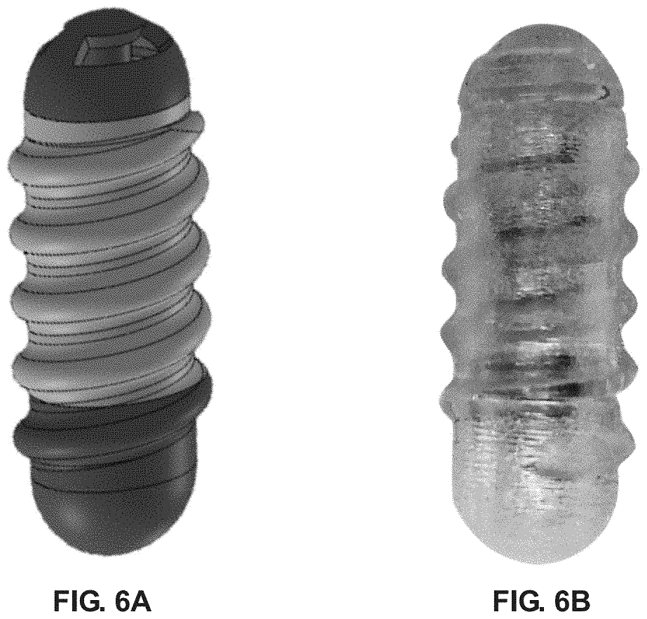

FIG. 6A is a schematic illustration of an ingestible capsule having a protruding helical microtextured pattern on its outer surface. The helical microtextured pattern provides a screw-like motion to facilitate a directional turning and churning motion. FIG. 6B shows a photograph of the capsule in FIG. 6A. The length, width, and angle of the helix may be varied. For example, the width of the helix may be about 0.2 mm to about 8 mm, preferably about 0.5 mm to about 2 mm, more preferably about 1 mm. The pitch of the helix may be about 2 mm to about 9 mm, and preferably about 4.5 mm. The helical pattern may be right-handed or left-handed.

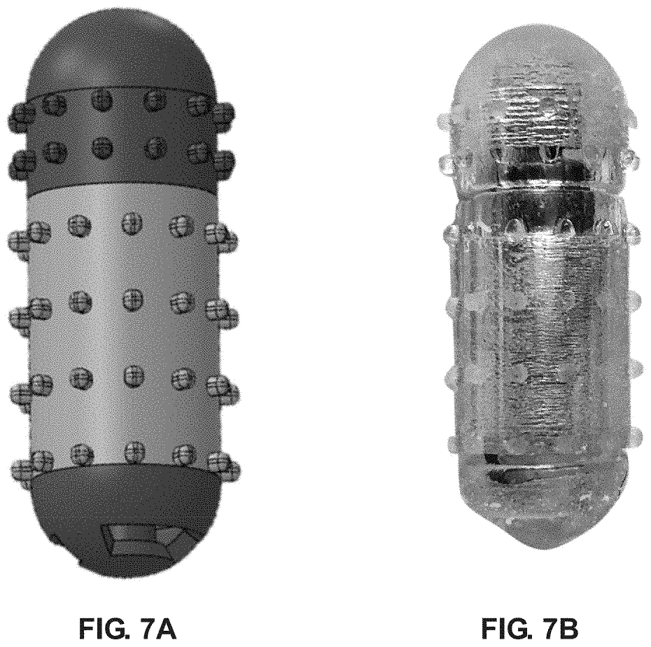

FIG. 7A is a schematic illustration of an ingestible capsule having a studded pattern on its outer surface. The studded pattern (also called protruding nubs) digs into and churns the mucosal layer. FIG. 7B shows a photograph of the capsule in FIG. 7A. The length of the studs extending out from the surface of the capsule may be about 200 μm to about 1200 μm. For example, in a version of the capsule used to wick mucus, studs with a length of about 700 μm to about 900 μm are preferable. The distribution of studs and the number of studs may also be varied. The studs may be distributed radially at 30- to 60-degree increments (e.g., 30 degrees, 45 degrees, or 60 degrees). The studs may be axially distributed at 1 mm to 6 mm increments. Stud patterns may be combined with any other microtexturing pattern. For example, the capsule may have an intruding helical pattern with studs patterned on the intruding helix surface.

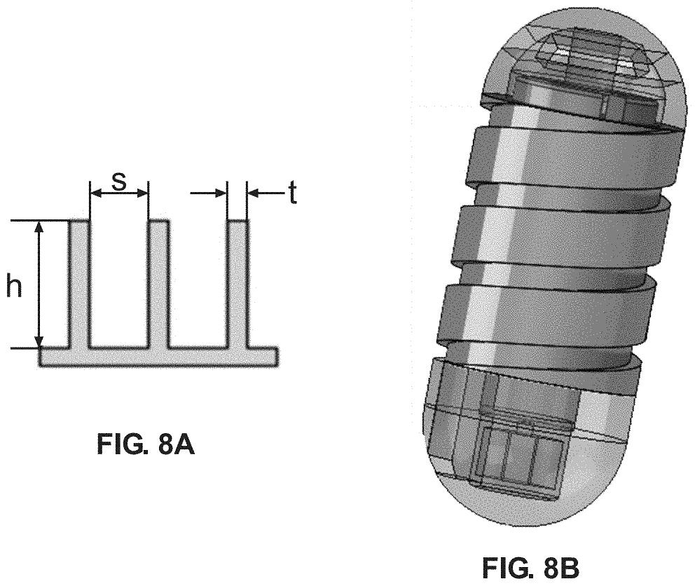

FIG. 8A is a schematic illustration of a cross-section of an intruding helical pattern on the outer surface of an ingestible capsule. FIG. 8B is a schematic illustration of the ingestible capsule having the intruding helical pattern in FIG. 8A. The intruding helical pattern (also called a spiral pattern) serves to reduce contact surface area, thereby reducing friction on the capsule that would counteract the capsule's rotational movement. The length, width, and angle of the helix may be varied. The pitch of the helix may be about 2 mm to about 9 mm, and preferably about 4.5 mm. The width of the helix may be about 0.2 mm to about 4 mm. The depth of the helix may be about 0.1 mm to about 1 mm. The helical pattern may be right-handed or left-handed.

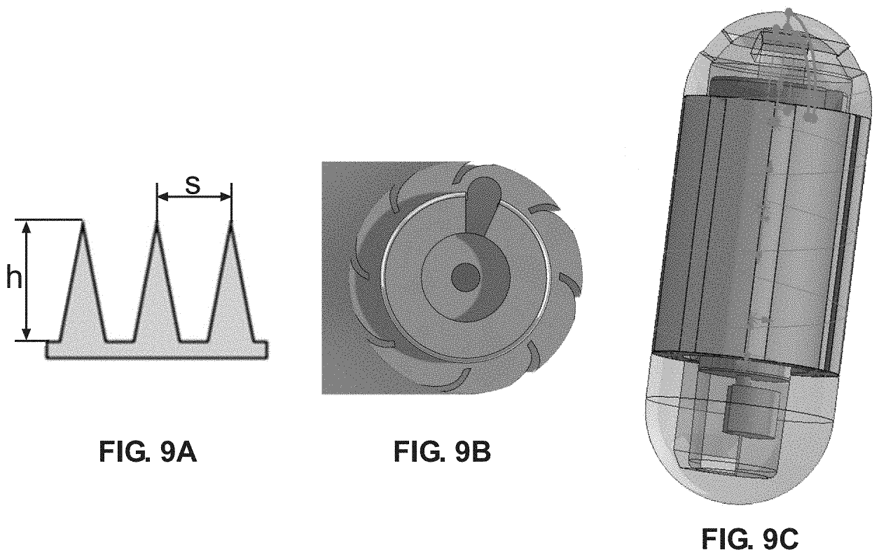

FIG. 9A is a schematic illustration of a cross-section of another intruding helical pattern on the outer surface of an ingestible capsule. FIG. 9B is a schematic illustration of the cross-section of an ingestible capsule having the helical pattern in FIG. 9A. FIG. 9C is a schematic illustration of the ingestible capsule having the helical pattern in FIG. 9A. The fin turbine pattern shown in FIG. 9C provides a churning motion. Like the helical pattern in FIG. 8A, the length, width, and angle of the helix may be varied. Unlike the helical pattern in FIG. 8A, the pattern in FIG. 9A has sawtooth points instead of flat edges that create points of friction that help the capsule rotate and stroke mucosa.

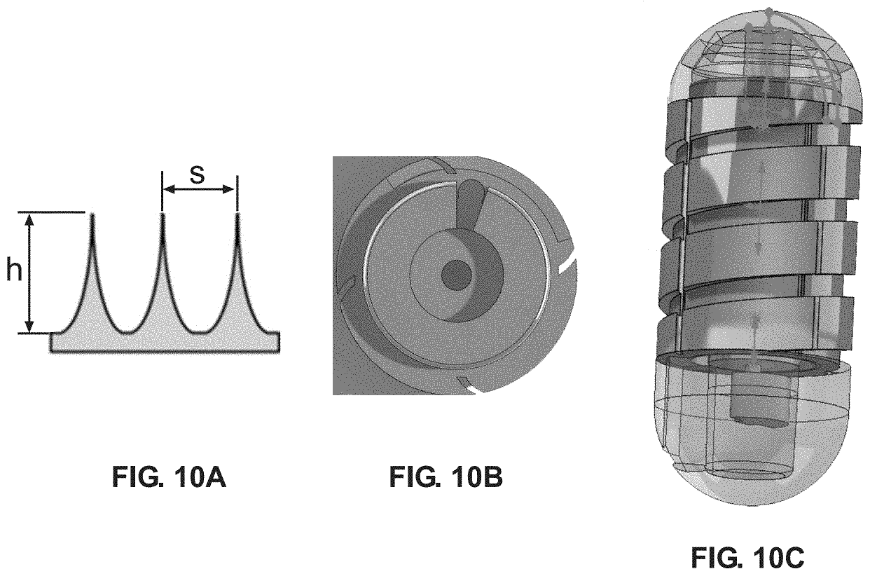

FIG. 10A is a schematic illustration of a cross-section of another helical pattern on the outer surface of an ingestible capsule. FIG. 10B is a schematic illustration of the cross-section of an ingestible capsule having the helical pattern in FIG. 10A. FIG. 10C is a schematic illustration of the ingestible capsule having the helical pattern in FIG. 10A. The helical pattern shown in FIG. 10C provides a combination of benefits, including reducing contact surface area to reduce friction that would counteract the capsule's rotational movement, and providing a churning motion. Like the helical pattern in FIG. 8A, the length, width, and angle of the helix may be varied. Unlike the helical pattern in FIG. 8A, the pattern in FIG. 10A has scalloped points instead of flat edges that create points of friction that help the capsule rotate and stroke mucosa.

The capsule may include a coating disposed on the surface of the housing. In versions of the capsule that include microtexturing, a coating may be disposed over the microtextured surface to cover these features. In this way, the coating may promote safe and comfortable capsule swallowing and passage through the gastrointestinal tract. The coating may degrade or dissolve in a fluid of a set pH in the same way as the membrane in the actuation assembly. The set pH may include any of the ranges described above with respect to the membrane. The coating may be formed from any of the materials described with respect to the membrane. In a version, the coating is the same material or materials as the membrane so that the coating and membrane both dissolve and/or degrade in the desired region concurrently.

Exemplary Capsule Inducing Illusory Satiety to Treat Obesity

Effective therapies for obesity either require invasive surgical or endoscopic interventions or high patient adherence, making it challenging for the nearly 42% of American adults who suffer from obesity to effectively manage their disease. Gastric mechanoreceptors sense distension of the stomach and perform volume-dependent vagal signaling to initiate the gastric phase and influence satiety. The Vibrating Ingestible BioElectronic Stimulator (VIBES) is a luminal stimulation modality that can activate these gastric stretch receptors to elicit a vagal afferent response commensurate with mechanical distension. VIBES is an ingestible device that performs luminal vibratory stimulation to activate mechanoreceptors and stroke mucosal receptors, which induces serotonin release as well as yields a hormonal metabolic response commensurate with a fed state. VIBES can traverse the entire gastrointestinal tract and be passed safely and naturally. Over 108 meals, treatment with VIBES significantly and consistently led to diminished food intake (˜40%, p<0.0001) and minimized the weight gain rate in a swine model (p<0.03) as compared to untreated controls. Application of mechanoreceptor biology stands to transform the capacity to help patients suffering from nutritional disorders.

The obesity epidemic, affecting nearly 42% of U.S. adults, increasingly strains healthcare resources by increasing the incidence of comorbidities such as diabetes, hypertension, cancer, and heart disease. Given the difficulty of modifying behaviors, the cost, complications and invasiveness of bariatric surgery and endoscopic bariatric interventions as well as limitations in weight loss associated with pharmacologic therapies, there remains a pressing need for new methods that efficaciously decrease weight gain.

Vagal nerve signaling plays a critical role in satiation through a negative feedback loop in which anorexigenic neurometabolic secretions are released in response to food intake. Distension of the stomach by food contents is transduced by intraganglionic laminar endings (IGLEs), the most-prevalent type of vagal afferents innervating the gastric musculature, which sense contraction and distension. These stretch mechanoreceptors produce short-acting vagal afferent signals and increase neuronal activity in the nucleus of the solitary tract (NTS) where vagal afferents terminate and interact with reward, energy homeostasis, hunger, and mood circuitry. In turn the NTS triggers metabolic and neural anorexigenic signaling to yield feelings of hunger or fullness and alter food intake. Since this mechanism is primarily volume-dependent, as opposed to composition-dependent, (carbohydrates, proteins, fats, or saline), methods to manipulate gastric volume—intragastric balloons (IGB)—were developed as an easy-to-deploy tool to minimize weight gain.

IGBs are designed to induce stomach distension to induce early satiety. Although they enable short-term weight loss during the adaptation phase, IGBs fail to promote sustained changes in hunger or eating behavior after 10-12 weeks nor do they demonstrate superior outcomes compared to pharmacologic or surgical therapy. Neural adaptation to the chronic distension (as opposed to periodic distension that results from eating), as well as placement, removal, perforation and obstruction complications pose challenges for the long term efficacy and safety of IGBs. Following numerous deaths in patients with IGBs since 2016, the FDA has issued warnings and some companies have recalled their IGB products.

Intervening more proximally, vagotomies and electrical vagus nerve stimulation (VNS), which are more localized interventions, have demonstrated preclinically to be associated with decreased weight gain, food intake, and sweet cravings, and increased satiation and energy expenditure. When clinically implemented for depression and epilepsy, VNS has shown a decrease in weight gain, reduced sweet cravings and increased energy expenditure. However, non-gastrointestinal (GI) side effects resulting from nonspecific axonal targeting at the cervical level of the vagus and metabolic compensatory mechanisms have prevented widespread clinical implementation for obesity. Vagatomies have also demonstrated significant benefit, although the mechanism and side effects are unclear and requires an invasive surgical procedure. Fundamentally, with the current technology and understanding of neural signaling, VNS systems cannot specifically target the relevant axons nor perform patterned stimulation to recapitulate the complex physiological signaling underlying satiety.

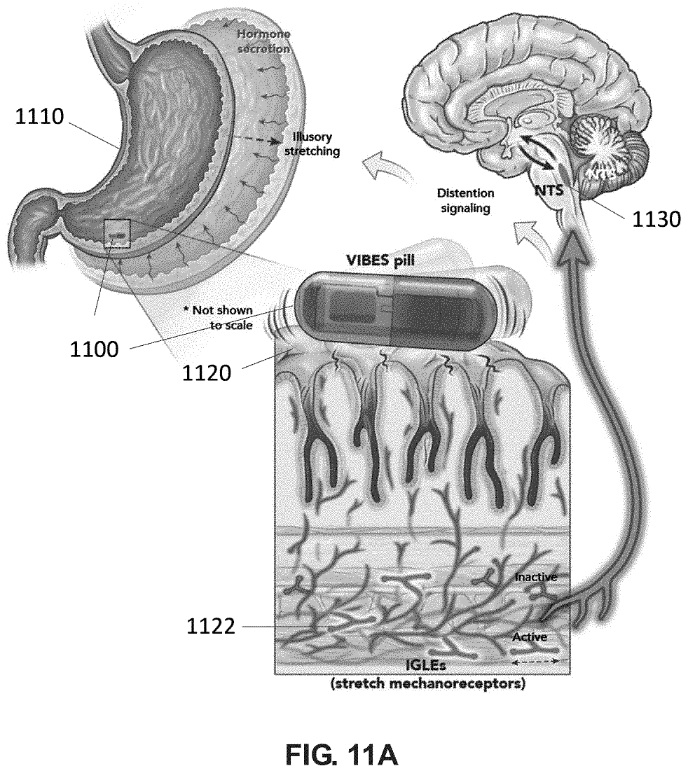

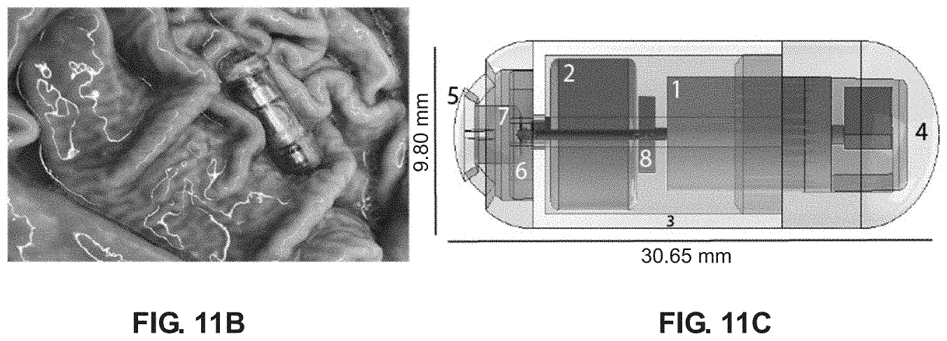

Considering the central role of gastric mechanotransducers in vagal neurometabolic satiety signaling, a mechanism and device capable of selective mechanoreceptor activation would pose significant value. Seminal experiments in stretch-sensitive spindle fibers in skeletal muscle have shown that vibration elicits illusory distension. Following a parallel mechanism, a vibratory stimulation modality was devised to selectively activate gastric stretch receptors and characterize their response. It was hypothesized that optimized intraluminal vibration of the gastric smooth muscle would induce illusory distension of the stomach, generating vagal afferents signals and a metabolic response commensurate to those elicited from mechanical distension in a fed state (FIG. 11A). The Vibrating Ingestible BioElectronic Stimulator (VIBES) pill, a safe, easy-to-use, ingestible device that can perform temporary, targeted, intraluminal stimulation prior to meals to achieve early satiety was then developed. In an awake and freely moving swine, it was hypothesized that the VIBES would reduce food intake and minimize the rate of weight gain as compared to untreated controls.

Design and Characterization of the VIBES Pill

The VIBES was designed to be orally ingested, to sustain contact with the gastric lining, to activate upon submersion in gastric fluid, to vibrate with amplitudes sufficient to stimulate gastric IGLEs for a set time period, and to pass safely through the GI tract. Its triple-zero capsule houses a gelatinous membrane which dissolves in 4.3+/−1.2 minutes following immersion in gastric fluid, releasing a spring-loaded pogo pin that completes the circuit to activate the vibrating motor. A motor with an offset shaft is positioned within a custom housing enabling displacement amplitudes of 2-4 mm when powered with a 1.55V 80 mAh silver oxide battery.

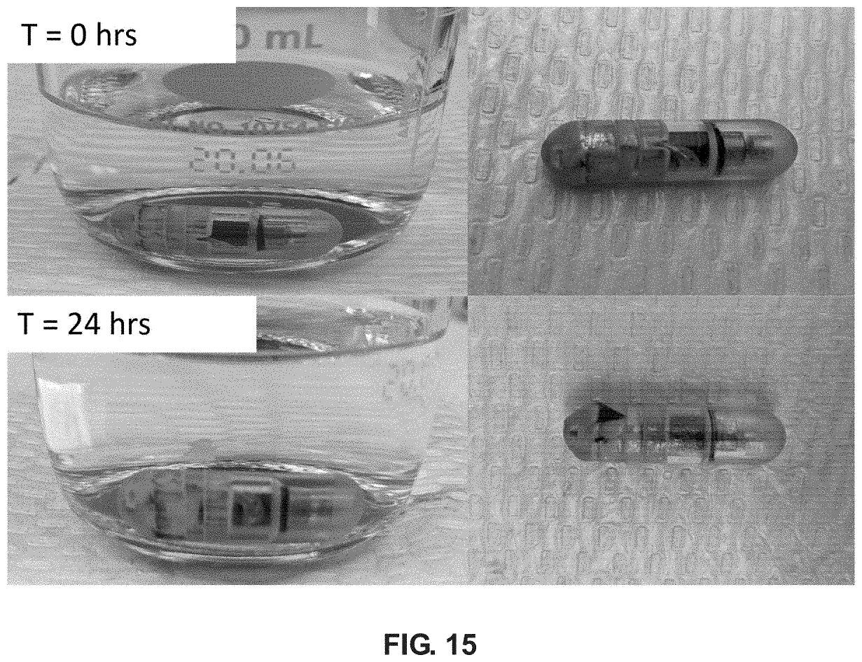

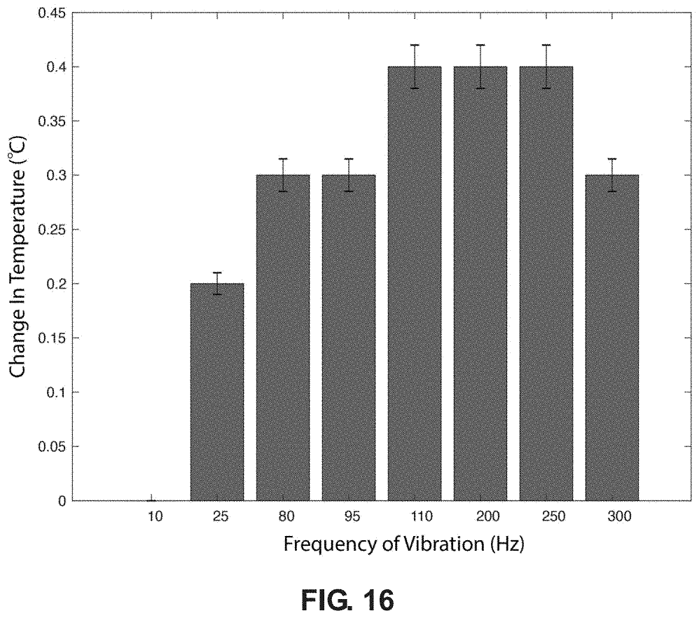

Duration testing was performed by immersing the VIBES pill in gastric fluid on a soft substrate. The pill actively vibrated for an average of 38.3±1.83 minutes for the VIBES pill (n=5). Since meals are generally consumed in a 20- to 30-minute window and gastric contents undergo primary mixing in approximately an hour, this time range was determined to be acceptable. To ensure that the system did not degrade or have vulnerabilities, a chemical resistance test (described below) was performed by immersing the VIBES pill in gastric fluid for 24 hours at 37° C. No macro or microscale changes were observed following immersion and pills were able to be successfully activated (FIG. 15). Thermal testing was performed to assess any potential heating risks on the surrounding tissue environment. 30 minutes of operation at various frequencies yielded less than a 0.5° C. change in the surrounding fluid, ensuring no thermal risks for the mucosal layer during operation (FIG. 16).

A swine model (50- to 80-kg Yorkshire pigs ranging between 4 and 6 months of age) was utilized to study the VIBES performance as its gastric anatomy is similar to that of humans. Further it has been widely used in the evaluation of biomedical gastrointestinal devices. Localization of the VIBES pill was characterized through endoscopic observation in n=5 swine. VIBES was endoscopically deployed into the gastric cavity and the final positioning was observed through the video channel over the course of 30 minutes. In all 10 trials, given its density of 2.019 g/cm³, the pill sank through gastric contents (densities 0.011-1.158 g/cm³) and achieved contact with the lining. Density of the pill was calculated using the properties of the following components: Masses: Pill capsule: 1.17 g; Motor (6×10): 1.6 g; Battery: 1.2 g; Pogo pin: 0.03 g; Wire: 0.3 g; Volume (11 mm diam pill)=2.13 cm³; and Pill density: 2.019 g/cm³. Localization in the gastric antrum and cardia were common and dependent on the positioning of the animal. In several cases, over the course of 30 minutes of stimulation, the pill was seen to migrate along the lining. In all cases, the VIBES was not emptied from the stomach by phasic inter-digestive migrating motor complex (MMC) for at least 30 minutes following administration. Thus, if VIBES is taken prior to meals to start the distension response, it should remain present to interact with the gastric lining during intake and the primary mixing phases of digestion.

Stretch-Receptor Activation to Signal Gastric Distention

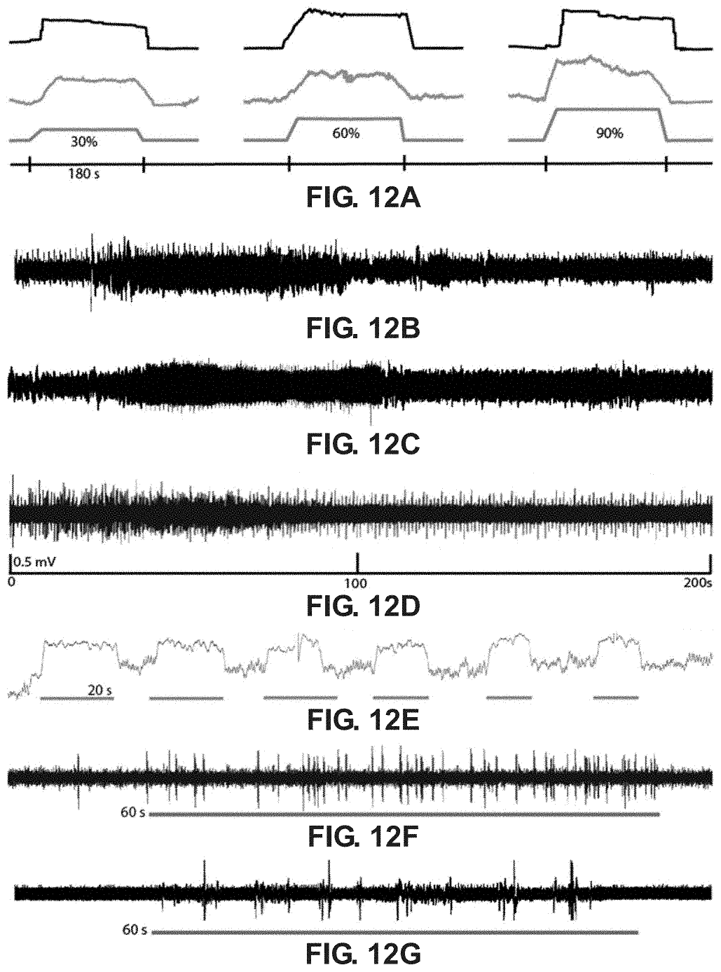

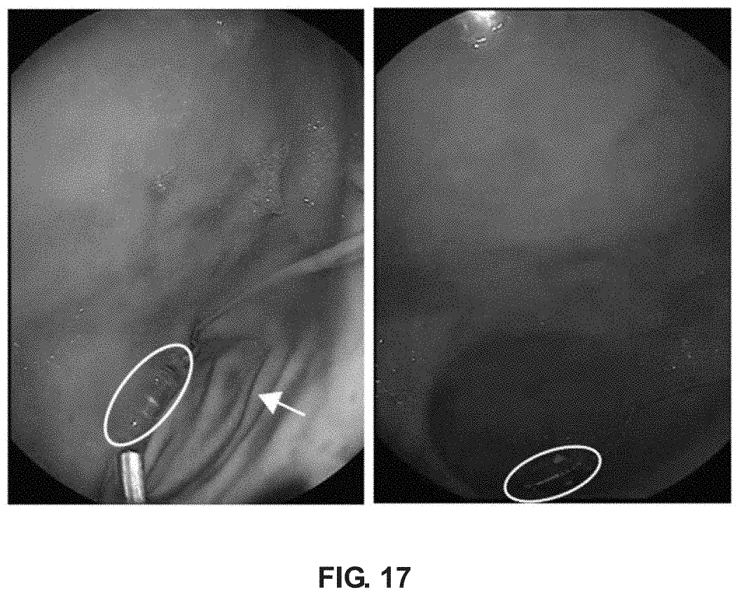

To characterize the neural signaling patterns of the stomach in response to mechanical distension and vibrational stimulation (n=4 animals), fine-wire electrophysiological recordings of up to 24 branches of the celiac vagus nerve, which innervate the gastric cardia, were performed following laparotomy. The celiac vagus was selected to isolate signals arising from the stomach and mitigate off target signals from other organs. Baseline recordings demonstrated small amplitude, spontaneous spikes along with slow wave related spiking. To mimic the distension created by the intake of food, the stomach was inflated using the endoscope to 30%, 60%, and 90% of its maximal volume and held for 180 seconds (FIG. 17). Five minutes of rest were provided between each insufflation state to allow neural activity to return to baseline. Spiking was observed in a subset of 6-10 channels with onset occurring 10-12 seconds following the beginning of inflation (FIG. 12A); these channels were designated as channels corresponding to axons innervating low-threshold stretch-sensitive IGLEs, consistent with prior studies. Spiking amplitude was higher and more densely concentrated at the onset of stretch followed by a slow adaptation rate (levelling off of distention-based afferents as the stomach adapts), commensurate with prior reports of these afferents (FIG. 12B). Upon desufflation, spiking acutely decreased and terminated within 2-18 seconds (FIG. 12A,). The firing rate was, on average, 118 Hz±22 Hz (n =6). The rate of firing graded monotonically with distension which is commensurate with the known spiking patterns of IGLEs. The distension was modulated by varying the volume of insufflation in the stomach.

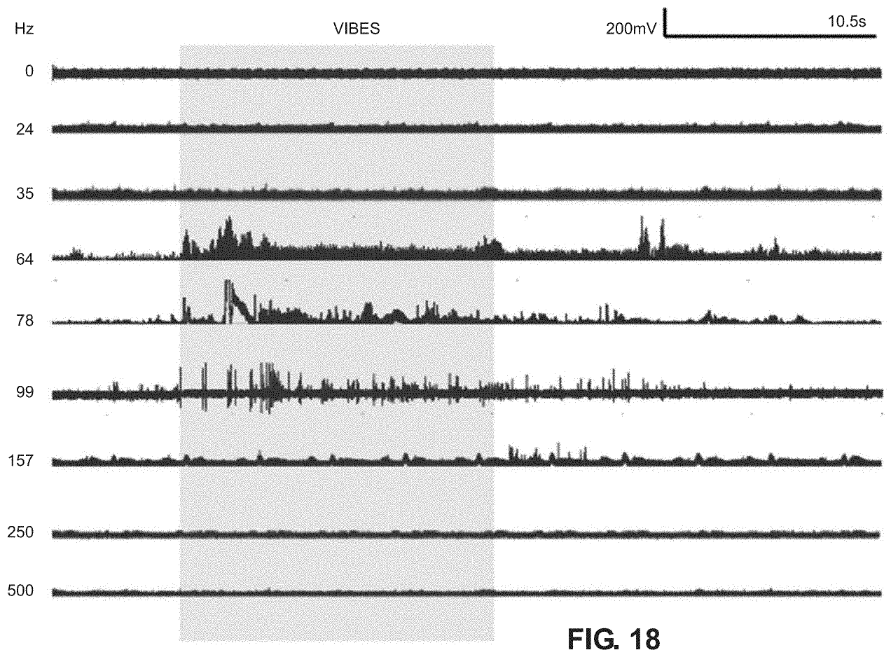

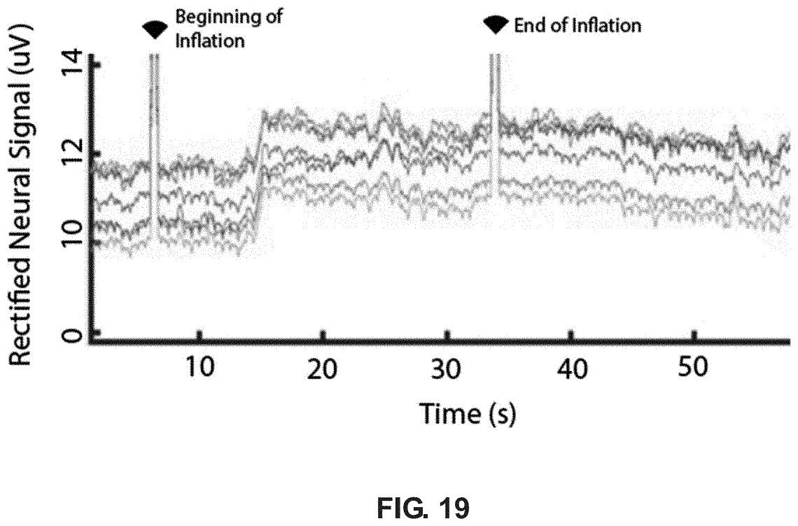

To determine if vibratory stimulation could activate these IGLEs to produce a similar response, neural activity on the channels that were active for stretch-sensitive IGLEs were monitored as vibration with frequencies between 24 Hz and 500 Hz was applied using the VIBES to the gastric lumen (FIG. 18). Spiking was observed at frequencies between 64 Hz and 100 Hz in a similar pattern to those observed during mechanical distention (FIG. 12A, FIG. 12C). At these frequencies, the amplitude of displacement of the pill was greater than 1 mm. No activation was observed for displacements under 1 mm, or for frequencies below 64 Hz or above 150 Hz. Onset of activation occurred within 2.5-3.0 seconds with slow adaptation, similar to mechanically-activated afferents. The plateau of the adapted region was 87-94% of the maximal rectified amplitude at onset (FIG. 19). Bilateral vagotomy was then performed and the above results were replicated to eliminate potential confounding between efferent signals and feedback loops. Raw spiking patterns from (FIG. 12B) mechanical insufflation (FIG. 12B), VIBES (FIG. 12C), and VIBES after bilateral vagotomy (FIG. 12D) demonstrate a high degree of similarity in shape, pattern, and frequency. Furthermore, spiking was able to be elicited repeatedly (FIG. 12E), without significant reduction in the onset or plateau amplitude (p<0.05, n=10). These results suggest that gastric luminal vibration induces afferent neural activation of IGLEs characteristic of spindle-type reception of distention.

Gastric Mucosal Stroking

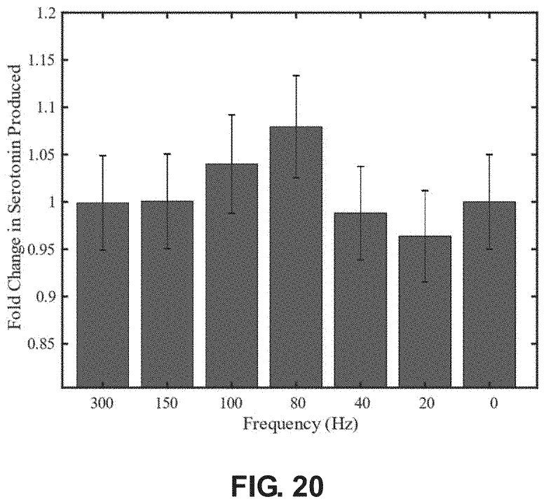

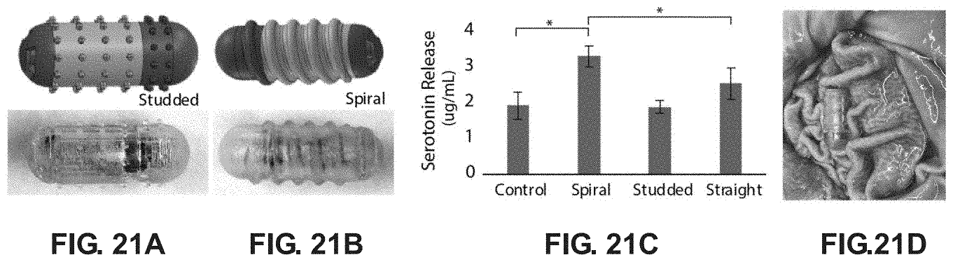

In addition to distension, mucosal stroking triggers gastric mechanoreceptors to stimulate gastric secretory activity. Gastric mucosal stroking was conducted via an endoscope using a thin filament that is known to elicit spiking for such receptors (FIG. 12G). During VIBES treatment, in channels not activated by stretch, such periodic bursting was observed (FIG. 12F). The VIBES pill's rotation, in response to surface friction and geometry constraints of the gastric mucosa, as it stroked the mucosa resulted in short, periodic bursts from these quickly adapting mucosal receptors. Mucosal stroking is known to release 5-HT or serotonin, which acts on vagal 5-HT3 receptors that perform satiation signaling as well as enteric 5-HT4 receptors which regulate peristalsis, secretion, vasodilation and digestion through intrinsic central and peripheral reflexes. The luminal secretion of serotonin in response to VIBES was assayed using ex vivo tissue on a Franz cell apparatus. 80 Hz resulted in the greatest increase in secretion levels as compared to the control condition of 0 Hz (FIG. 20). Based on this data, and the inconvenient human audibility of the VIBES pill above 100 Hz, 80 Hz was selected as the optimal operating frequency. Additionally, surface geometries were altered to enable greater stroking of the mucosa (FIGS. 21A-21D), including studs and ridges to enhance surface contact. A spiraling design induced a significant increase in luminal serotonin release as compared to the straight surface and control (untreated) conditions.

Metabolic Effect of VIBES

To characterize the downstream effects of VIBES through vagal afferent signaling, the hormonal secretions relevant to feeding behavior and satiety were profiled. Blood was sampled at 0, 15, 30, 45, 60, 90, and 150 minutes for animals receiving a VIBES pill (n=6, between 30-60^{th} minute marks) and for no treatment (n=6). While the control animals exhibited the expected levels of these hormones in a fasted state, treatment with VIBES resulted in a significant reduction of ghrelin, the ‘hunger hormone’ (p<0.01). Ghrelin decrease is normally a postprandial response but occurred here in fasted animals treated with VIBES. Furthermore, insulin levels increased significantly (p<0.01) with an amplitude and rate commensurate with normal meal ingestion. Glucagon, responsible for maintaining blood glucose levels, increased until minute 60 and then decreased as time progressed. Levels of C-peptide, associated with the biosynthesis of insulin, GLP-1 which enhances insulin secretion, and Pyy, an appetite suppressant, increased significantly with stimulation (p<0.01). Together, these trends suggest that by signaling distention artificially, VIBES can induce the gastric phase metabolic response. Glucose from a venous catheter was measured at 30 minute intervals before and after administration of VIBES. No animals demonstrated hypoglycemia during the course of the study.

VIBES Reduces Food Intake

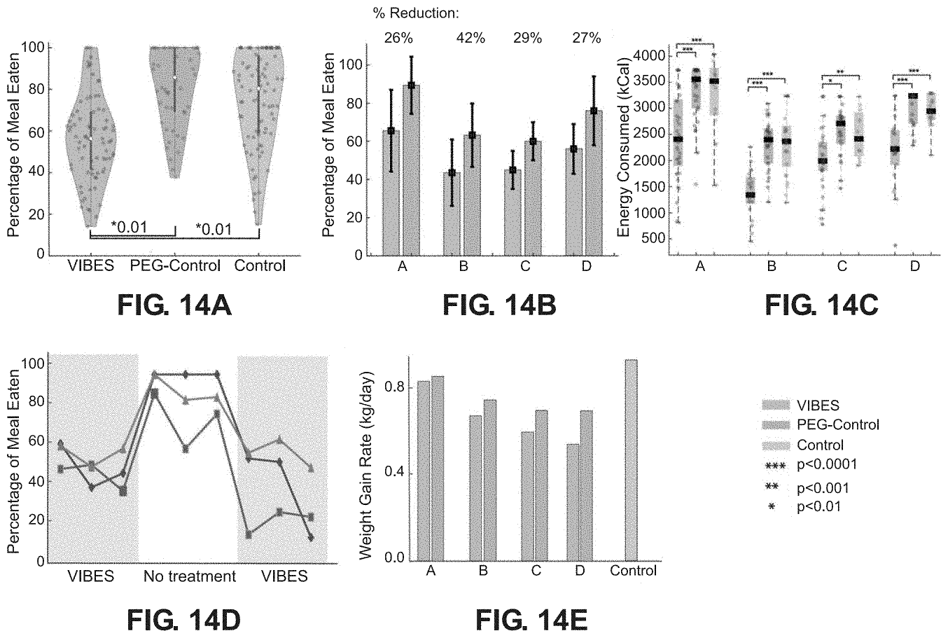

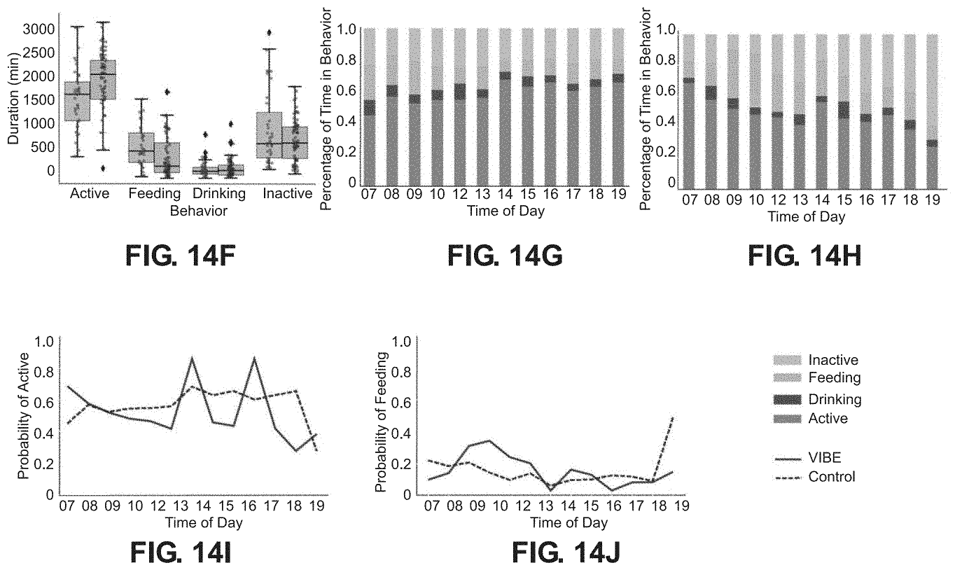

To investigate the effect of VIBES on hunger and eating behavior, the food intake of four swine was monitored for at least 24 meals with no treatment (PEG-control group) and when treated with VIBES tethered through a PEG tube for 30 minutes prior to each meal (VIBES group). Tethering was performed with a very flexible leash of 10-15 cm, enabling it to excurse around in the stomach freely. Endoscopic observation of the free and tethered VIBES demonstrated no significant difference in contact, vibration, mechanical force transmission and/or stroking of the gastric lining. Four animals matched in size and age were also monitored as controls, to account for potential effects of the PEG tube (control group). All animals were fed ad libitum with 2 meals (pellets) and one snack of 5 apples each day. Prior studies have demonstrated that satiety is well-marked by the amount of food consumed in animals. The average percentage and standard deviation of the meal consumed for the VIBES, PEG-control, and control group were 58.1±10.7 (n=108 meals), 84.1±4.5 (n=100 meals) and 78.4±4.5 (n=96 meals), respectively. Intake in the VIBES group was significantly lower than the PEG-control and control groups (p<10⁻²⁰, student's two-tailed homoscedastic t-test, FIG. 14A). There was no significant difference between the PEG-control and control groups (p>0.05), indicating the presence of a PEG tube itself did not confound the significant difference seen during VIBES treatment. On a per-animal basis, VIBES treatment resulted in significant reductions in intake (p<0.001 in all cases, student's two-tailed homoscedastic t-test), averaging 31% of the usual intake. Energy consumed per meal by each animal treated with the VIBES was also significantly lower than the control groups (FIG. 14C, p<0.001 in all cases, student's two-tailed homoscedastic t-test). No adaptations or trends in intake were observed over the 24-meal period. To assess latencies and potential long-lasting effects of the treatment, a cross-over study design was utilized in which swine were treated for three meals, untreated for the subsequent three meals, and treated for the final three meals (FIG. 14D). Intake sharply increased during the untreated window, suggesting that VIBES functions through temporal vagal activation, with little neural adaptation or long-term effect. The weight gain rate during the VIBES treatment period was significantly lower than the control period (p<0.03, student's two-tailed paired t-test, FIG. 14E) and significantly lower than the control group (p<0.01, student's two-tailed heteroscedastic t-test). Together, these data suggest that the VIBES pill significantly decreases food intake and slows the rate of weight gain in a large animal model.

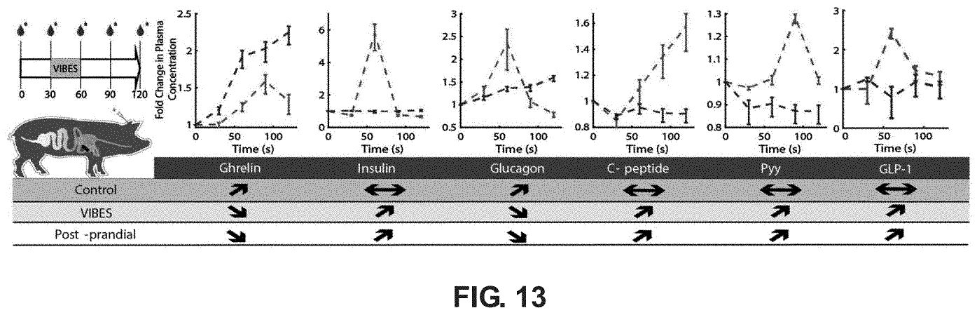

A preliminary behavioral study was conducted using an image-based deep learning and statistical model trained on twelve continuous hours of labeled daytime data (7a -8p). This was used to analyze 96 hours of unlabeled daytime video data from four pig subjects in either the treatment or control conditions. The time spent within each of four behaviors, detailed in Table 1 in terms of occurrences, durations, and probabilities within the control and treated conditions, was analyzed. The average percentage of time spent in each behavior demonstrates a trend towards more time spent in and over the feeder in the treatment condition (FIG. 14F). Although the length of feeding bouts are not correlated with satiety, they may be a marker of adaptive behavior to the intervention. Further, stimulated animals slightly trended towards more inactivity. On an hourly basis, controls demonstrated a relatively consistent level of activity and feeding throughout the day, while both trended downward in the treatment group (FIGS. 14G, 14H). Such reductions in physical activity and foraging have been reported to reflect postprandial satiety in laboratory pigs. As treated animals consumed less food, on average, it was hypothesized that they may demonstrate more appetitive behaviors before or near mealtimes, corresponding with a higher drop in blood glucose levels. These behaviors include locomotion correlated with foraging, rooting and interacting with cage walls and are positively correlated with feed restriction. These behaviors were categorized in the active category and determined probabilities of a given behavior on an hourly basis. Peaks in the probability of active behaviors near mealtimes were observed, potentially indicative that treated animals, having consumed less food in a prior meal, were ‘hungrier’ for the next meal (FIGS. 14I, 14J). The probability of inactive behavior also trended downward in treated animals over the course of the day and could be related to compounding or prolonged postprandial satiety, which is correlated with stabilized insulin and glucose levels measured (FIG. 13).

TABLE 1
Behavior Classification.

| General | | |
|---|---|---|
| Category | Behavior Type | Behavior Description |
| Inactive | Sleeping | No movement, not standing, eyes open |
| | Resting | No movement, not standing, eyes closed |
| Feeding | Free feeding | Head is in food bin, chewing |
| | Pedialyte | Pedialyte through cage doors while in-vivo |
| | | team exchanges batteries/fixes bandages: |
| | | head touching bottle |
| Drinking | Water Spigot | Head to water spigot |
| Active | Hanging Toy Ball | Head or body touching the hanging ball |
| | Hanging gear | Head or body touching the gears |
| | Free moving toy | Interaction with head/body; moving |
| | Scratching/rooting | Pawing at or using their nose to move |
| | woodchips | woodchips |
| | Scratching or | Body up and touching the walls of the |
| | climbing | cage |
| | on walls of cage | |
| | Interaction with | Touching, nosing, being fed from |
| | researcher | researcher |

Safety and Biocompatibility

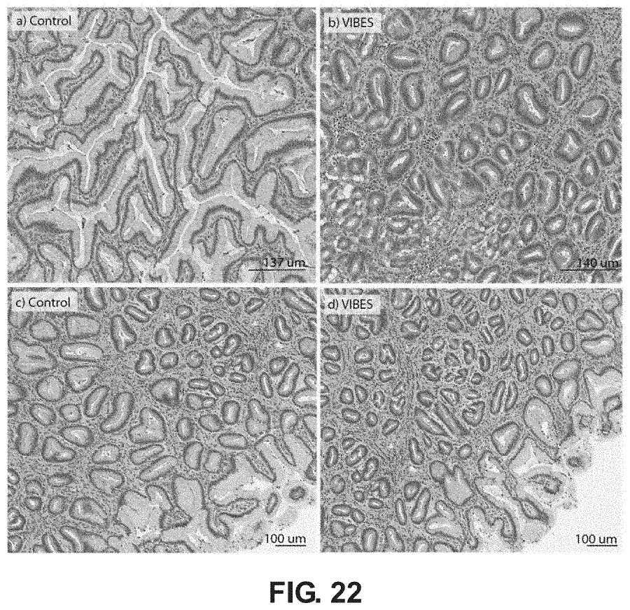

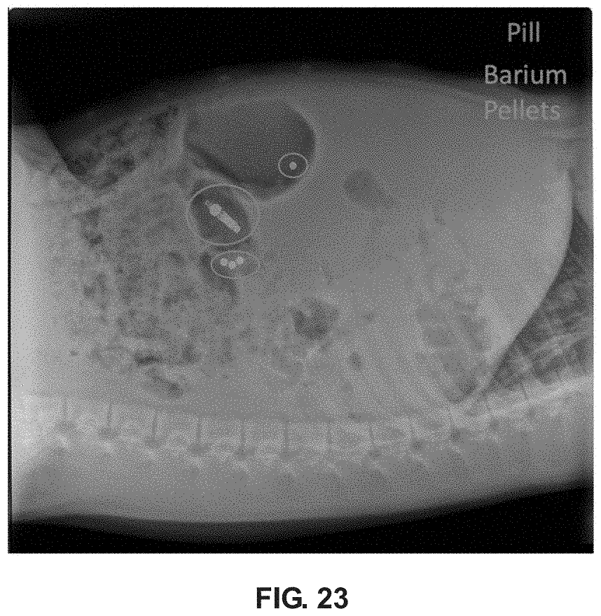

Following two weeks of daily VIBES usage (twice a day), no abrasion, irritation, or inflammation was observed on endoscopic examination of the gastric cavity. Further H&E staining on fixed sections of tissue following explant revealed no aberrant morphology, irritation or inflammation (FIG. 22). To assess potential effects on motility, animals were either fed a sham pill (n=3 swine) or the VIBES pill (n=3 swine) along with a set of small radio-opaque barium pellets. Radiography was performed every two days to determine the period for clearance of all barium pellets (FIG. 23). In animals with VIBES, swine passed all the pills in 4.3 days on average (range 4-5 days), while in control animals, passage took 8.3 days on average for sham pill passage (range 7-9 days) (Table 2). These data suggest that VIBES does not have any negative impact on motility. In all trials, animals were able to pass the pill without obstruction, perforation, or any signs of distress.

TABLE 2
Days for passage of the barium pellets for animals
orally administered a VIBES or sham pill.

| | Number of Days to Pass all Barium Pellets | |
|---|---|---|
| Animal # | VIBES | Controls |
| 1 | 4 | 9 |
| 2 | 5 | 7 |
| 3 | 4 | 9 |

As demonstrated by electrophysiology and the 5-HT response, the VIBES may activate 5-HT3 receptors, which trigger critical gastrointestinal functions such as pancreatic secretion, meal termination, early satiety, and appetite regulation. However, it is known that excessive activation of 5-HT3 results in nausea and vomiting. Thus, all animals were monitored during treatment by 4-6 staff periodically during the day and through continuous daytime and nighttime video recordings 24 hours of the day. No signs of distress or emesis, or diarrhea were observed in any animal.

Discussion

In this example, a modality of luminal vibratory stimulation that activates gastric stretch receptors to signal distension and initiate the gastric phase was established. By optimizing the range of vibrational frequencies and including features in the VIBES pill that increased mucosal interactions, not only were vagal afferents signals relevant for indicating distention generated, but a significant and consistent decrease in food intake in swine was also induced. Restriction of caloric intake during meals is a well-documented and sustainable mechanism to limit weight gain. The VIBES pill could be ingested on a relatively empty stomach 20-30 minutes prior to an anticipated meal to trigger the desired sensation of satiety early in the meal. Shaping this luminal stimulation modality into a pill format presents several valuable advantages over its alternatives. As an ingestible device, no invasive implantation or surgery is required. Stimulation can be performed directly in the gastric cavity with a triggered activation, making the stimulation specific to the tissue of interest.

Injection molding techniques and mass manufacturing of electronics coupled with natural passage makes the VIBES a consumable device, with no re-acquisition or recharging of the device. For certain patient populations, this enables temporary therapy, without the need for surgery. However, with improved power transfer and charging technologies, implantable or gastric-resident actuators could be developed to relegate repeated oral administration to patients requiring chronic therapy.

The robust design of the VIBES overcomes practical limitations common to human oral consumption/administration. The 30 Hz range of frequencies within which the stretch response enables the VIBES' s function amidst varying gastric contents which may dampen the induced vibrations to varying degrees. The location of the VIBES pill is not controllable and likely to shift during a meal. This does not impede its function in the stomach as receptors in the antrum and cardia are predominantly stretch-sensitive. Additionally, VIBES can consistently create a stretch response as gastric tension receptors are slowly adapting as compared to mucosal mechanoreceptors, which fire in short bursts as seen in prior studies. The large animal study demonstrates that over a two-week period, and despite variations in the animal's routine, sleeping patterns and activity, food intake was consistently lower when treated with VIBES. The response to the intervention also reinforces earlier studies highlighting the predominant influence of gastric distension in the satiety response.

Use of the VIBES resulted in no observable distress or negative side effects and normal passage in more than 20 trials in a large animal model, supporting the pre-clinical safety in a relevant animal model.

Weight loss studies are most commonly conducted in rodent models, given practical constraints of cost and resources, similarity in physiology, and ease of dietary-induced disease models. The choice of animal model was the swine, given the need for human-sized anatomy accommodating the geometric dimensions of the VIBES pill. However, weight loss is difficult to measure meaningfully in the young and growing swine species that are used for laboratory research.

Following further safety validations, clinical translation could facilitate a paradigm shift in potential therapeutic options for diseases such as obesity, polyphagia, and Prader-Willi syndrome in which late onset of satiety yields excessive overeating and subsequent metabolic, cardiac, and endocrine comorbidities. The metabolic response triggered by VIBES could also be leveraged to treat diseases of insulin insufficiency or dysregulation such as diabetes. Its ability to increase the motility rate should be further studied. Optogenetic activation of the IGLEs could be another way in which the receptor physiology in the stomach could be modulated.

Overall, this example lays the foundation for a new modality of vagal stimulation, acting through gastric mechanoreceptors, to induce an illusory sense of satiety, decrease food intake and limit the rate of weight gain, paving the way for a new treatment for obesity.

### Methods

Design and Modelling of the VIBES

The VIBES was modeled in Solidworks. The pill was designed to be the same dimensions as a triple zero capsule. The pill was designed with three sections that press-fit onto each other in order to create a tight seal, in which with two sections housing the electronics are completely sealed off from the third section, where gastric fluid is allowed to enter the capsule to dissolve the glucose layer and activate the pill. The vibration is achieved via an offset weight on the shaft of a DC motor. Due to its fabrication by 3D printing, a wall thickness of 0.6 mm was used in concordance with the capabilities of the Stratasys printer for the pill capsule's thickness. The 1.55 volt 80 mAh Silver Oxide battery (DigiKey) was used due to its biocompatibility and its high capacity to size ratio. A spring-loaded mechanism using a pogo pin (DigiKey) was designed to activate the pill once it reaches the stomach. The pill was designed to allow gastric fluid to enter through the pill cap and dissolve a glucose membrane separating the negative lead of the battery from the negative lead of the motor.

Fabrication of VIBES

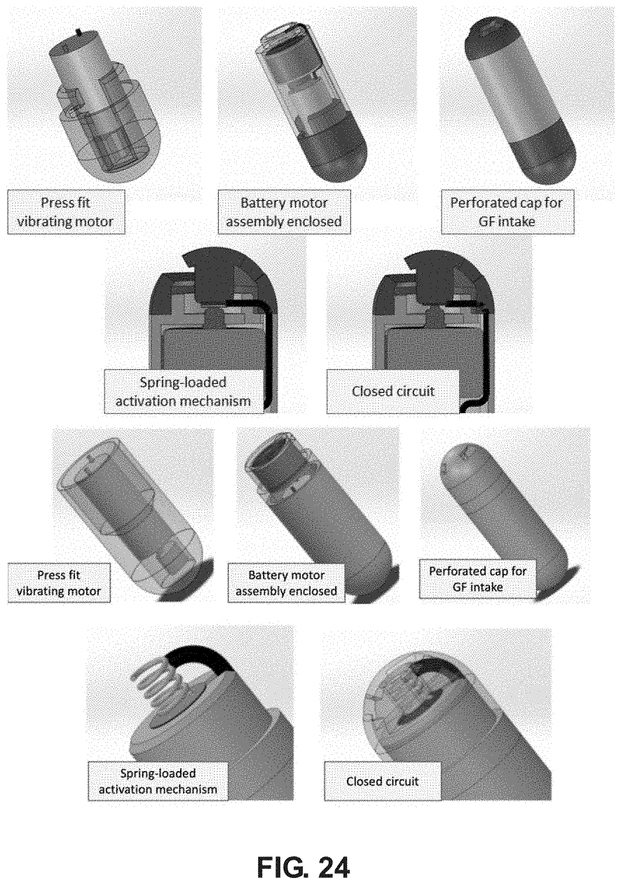

The outer capsule of the VIBES was 3D printed using the Stratasys VeroClear photopolymer due to its strength, transparency, and chemical resistance, making it the ideal material for prototyping. The pill is split into three sections: the motor cap, the central body, and the pill cap. To assemble, the vibrating motor was first pressed into the motor cap. A copper pad is soldered on the positive lead and affixed to the bottom of the motor. The spring-loaded pogo pin was then pressed into the central body, and the battery was inserted such that the negative terminal was in contact with the pogo pin. The central body assembly was then pressed onto the motor cap assembly such that the positive terminal of the battery contacts the positive copper pad on the motor. The negative lead of the motor was stretched through the central body to the pill capsule. The negative lead was soldered onto a conductive pad in the top section that, when fully assembled, closed the circuit by contacting the pogo pin. A glucose membrane was also placed between the negative lead pad and the pogo pin that is designed to dissolve once the pill is immersed or in contact with gastric fluid, allowing it only to be activated when it reaches the stomach (FIG. 24).

Pill Characterization (Vibration Time Test and Chemical Resistance Test)

The pill's vibration time was characterized using 80 mAh silver oxide batteries by measuring the amount of time it took for the pill to stop vibrating on a full battery. The chemical resistance of the pill was measured via a 24-hour submersion in simulated gastric fluid to determine the effects on the pill's casing and if the fluid would encounter the electronics on board.

Thermal Risk Testing

To evaluate the thermal safety of the pill, the heat output in water and air was measured using the FLIR A65SC Test Kit and a thermal black body (Dahua Technology JQ-D7OZ) as a calibration standard. The pill was vibrated at 25, 80, 95, 110, 200, 250, and 300 Hz for 30 mins each in a beaker with 20 mL of saline to determine the average, minimum, and maximum temperatures achieved at each frequency in the surrounding fluid.

Passage Safety

All animal experiments were conducted in accordance with protocols approved by the Committee on Animal Care at the Massachusetts Institute of Technology (MIT). All large animal studies were performed in a swine model (50- to 80-kg Yorkshire pigs ranging between 4 and 6 months of age). The swine model was chosen because its gastric anatomy is similar to that of humans and has been widely used in the evaluation of biomedical GI devices. Three female swine were administered the VIBES pill and monitored by X-ray imaging every other day until the pill was passed. Any changes in behavior, eating, or indications of pain or distress were monitored and recorded using a video camera placed near the cage (MIPC systems).

Electrophysiology

In terminal procedures, following laparotomy and dissection, in which the celiac branch of the vagus nerve, running alongside the left gastro-omental arteries, was identified, a set of 10-12 30 gauge fine wire electrodes were inserted into the celiac branch of the vagus nerve using 26 gauge needles. The stomach was inflated to differing degrees using the endoscopic air inflation/suction channel by monitoring the volume and flow rate of the air. Electrical signals were recorded using the Intan Technologies Recording and Stimulation System at 30 kHz. 20 minutes of baseline signals were recorded. Then, the stomach was inflated to differing degrees using the endoscopic air inflation/suction channel. The volume of the stomach was first determined by fully inflating the stomach, observing the separation of rugae and measuring the volume of air used. 30%, 60%, and 90% of these volumes and separation levels were utilized for a graded study of the neuronal spiking physiology. Then, another 20 minutes of baseline was recorded. The VIBES pill was then introduced into the stomach and electrical signals were recorded in a similar manner.

Data was exported and processed in MATLAB. Data was bandpass and notch filtered (60 Hz) to remove low frequency motion artifacts, breathing artifacts and white noise. The data was further rectified and adjusted based on the baseline of the mean rectified signal. The rectified data was smoothed using a 3000-sample moving window).

Serotonin Screening

A 6x4 well Franz cell apparatus was used to test mucosal serotonin release in ex vivo tissue from swine. VIBES treatment was applied for 20 minutes, and luminal secretions were sampled by washes of 200 μL and compared to that of untreated control tissue. 6 independent samples were collected at three time points. A serotonin ELISA (Enzo bio sciences) with fluorescent readout was used to quantify the secretions.

Metabolic Panel

A metabolic hormone panel (Eve Technologies) was run on venous blood from an ear vein catheter, collected 30 minutes prior to stimulation and every 30 minutes for 2 hours or in animals without any stimulation in awake and freely-moving animals (n=6). Collection vials spray coated with EDTA were inverted five times each. Blood was treated with a protease inhibitor cocktail (S8830, Sigma) within 10 minutes of collection and centrifuged for 15 minutes at 4° C. at 4000 rpm. Blood analyzed for GLP-1 was treated with DPPIV inhibitor (Sigma) within 2 minutes of collection and centrifuged for 15 minutes at 4° C. at 4000 rpm. ELISA results were normalized to the baseline values. Control samples were run with every assay run to ensure that all assay signals of a particular lot fall within an acceptable range.

2-Week Feeding Study

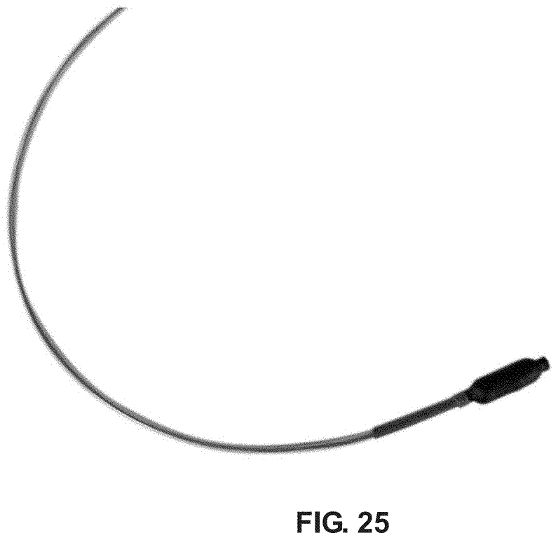

A tethered pill (FIG. 25) was inserted through a 28-gauge percutaneous gastrostomy (PEG) tube (AVANOS) that was placed with endoscopic assistance. Daily stimulation was then performed for 30 minutes 20-30 minutes prior to mealtimes at 7:30 am and 3:30 pm. The animal was fed Labdiet mini-pig grower pellets (5081) at 7:30 am and 3:30 pm and a snack of five apples between 11 am and 12 pm. The mass of the food provided, and leftover in the hopper (or anything spilled nearby) after 30 minutes was measured. Videography of stimulation and feeding periods was analyzed to surveil for any changes in behavior, appetite, and side effects such as nausea, vomiting, lethargy, etc. The animal's body weight was measured at least twice per week and fresh fecal samples were collected twice a week after stimulation.

Image-Based Behavioral Study

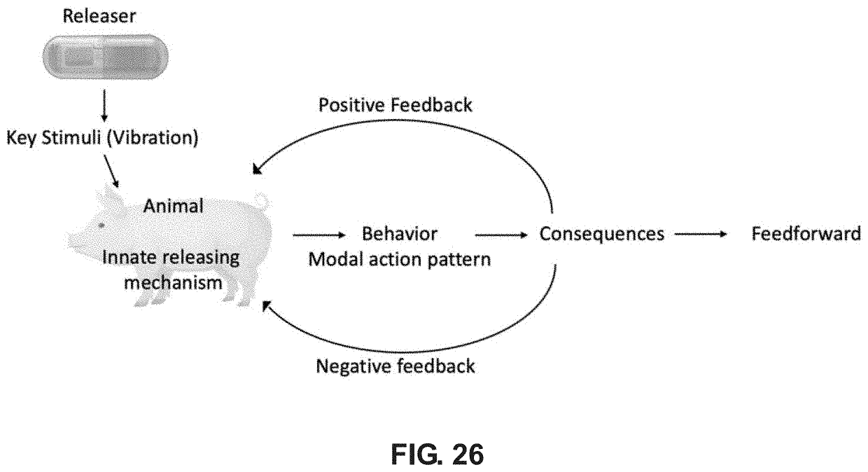

To capture trends and insights of the behaviors exhibited by the subjects in the control and treated conditions, a deep learning architecture was developed. Prioritizing interpretability, Lehner' s ethological model of innate behavior was drawn on to formalize the problem of classifying behavior and chains of behavior (FIG. 26). Vibration is the stimulation being given by the VIBES, the releaser, to the animal prior to food ingestion. VIBES may then influence eating, drinking, and activity levels. Within operant condition behavior theory, the consequences of satiation have neither positive nor aversive reinforcing value; the feedback mechanism from consequences is time-dependent. Initially, feeding is a positively reinforcing stimulation; however, the value of the reinforcer decreases over time until the animal reaches satiety. If the animal continues to eat, feeding becomes a negatively reinforcing stimulation, increasing in negative value as the animal continues to feed. The next behavioral act in the continuous stream of behavior can be elicited by the consequence of the behavior that precedes it.

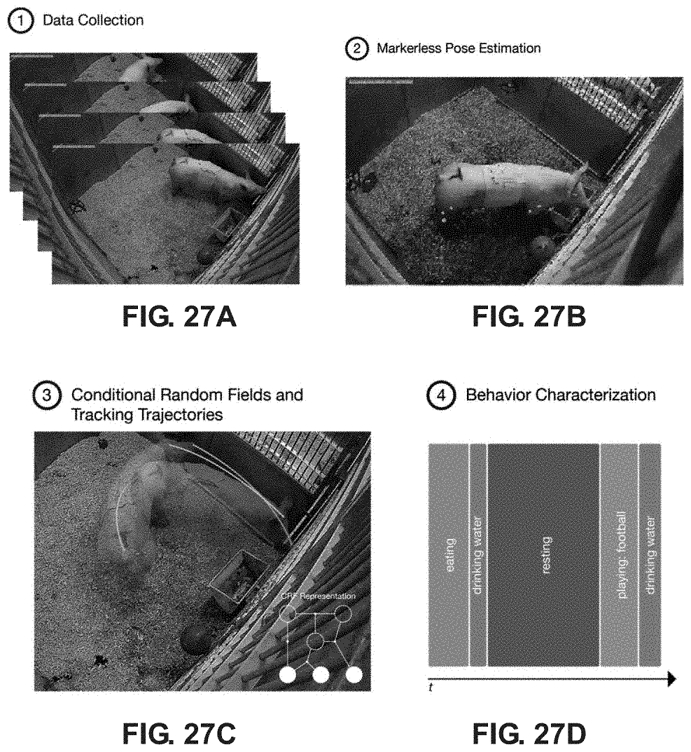

To classify the behaviors exhibited by the subjects and track differences between conditions over time, audio and video data of the pigs in the animal facilities was collected using 1080 p HD IP (network) cameras with night vision and audio mounted in the corner at the top of the pen (FIGS. 27A-27D). Animals were held in woodchip embedded pens (5′×5′) with toys, water spigot, and food supply fixed to the walls. Continuous 1920x1080 resolution RGB video data at a rate of 15 FPS, and audio data at 16000 Hz were collected. The data from 4 pigs in 2 pens for 6 non-continuous days was collected for a total of 94 hours.

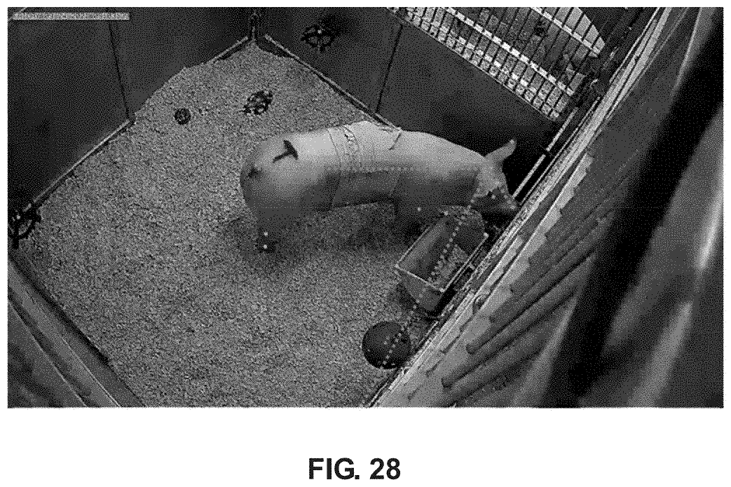

Markerless pose estimation (FIGS. 27B and 28) was performed on raw video data with 2D pose integration. A model was trained to capture time-series estimations for the positional coordinates of 22 different body parts and 10 objects in the environment. This incorporated a deep convolutional neural network that has shown inter- and intra-species transfer learning capabilities. The model was trained on a dataset consisting of 420 frames over 12 hours of daytime video. Three independent video coders annotated 420 image frames were labeled spanning a variety of perspectives and colors to help increase performance. The set of image frames were randomly sampled while preserving representation of the distinct view angles, variety of illumination levels, and different resolutions and video qualities. To verify label consistency, a 10-pixel radius was generated around the original labels and Crohnbach's alpha was calculated to measure the inter-rater reliability between video coders, resulting in a score of 0.837 and demonstrating a high internal consistency of labels.

Twenty-two different key points comprised of relevant pig body parts and objects in the environment were labeled (Table 3).

TABLE 3
The feature set for the key point model.

| Animal/ | |
|---|---|
| Object | Body Part Key points |
| Pig | Front Legs (elbow joint, carpal, and coffin), |
| | Rear Legs (stifle joint, tarsal, and |
| | coffin), Left and Right Scapula, Apex of Left and |
| | Right Ear, Head, Eyes, Snout, |
| | Proximal and Distal Tail Locations |
| Hopper | 4 corners of the top of the hopper |
| Water | Spigot mouth |
| Spigot | |
| Toys | Center of the tethered and free moving toys |

For body part tracking DeepLabCut (version 2.2.1.1) was used. Specifically, 420 frames taken from 21 videos/animals were labeled (then 95% was used for training). A ResNet-50-based neural network with default parameters was used for 400,000 training iterations. This was validated with one shuffle, and found the test error was: 6.34 pixels, train: 9.58 pixels (image size was 1920 by 1080). Then, a p-cutoff of 0.6 to condition the X,Y coordinates for future analysis was used. This network was then used to analyze videos from similar experimental settings.

The positional coordinate time series output from the DeepLabCut model was used to estimate the behaviors exhibited by the pigs. A more statistical-based machine learning model called Conditional Random Fields was used to estimate the pig's behaviors during a window of time.

A single day's worth (24 hours) of video data from each participant was coded using the ethograms below. The primary coder was blind to experimental conditions until after analysis. Secondary coders were not blind to the experimental conditions. A strong inter-rater reliability between coders was reported (κ=0.92). The data generated from these labels was used to train a deep learning model to automatically detect activities/behaviors using markerless pose estimation and location.

The data output from DeepLabCut was a time-series positional coordinates dataset paired with activity labels and a series of images. From here, Conditional Random Fields was used to infer latents and states. This allowed features such as time, frequency, duration, and trends of behaviors across days to be calculated.

Twelve behaviors, such as sleeping and eating from the feeder (Table 4), were classified. Classes are imbalanced with as many as 284,004 data points for ‘resting’ and as few as 2,103 for ‘being fed Pedialyte.’ Conditional Random Fields were used to perform classification over temporal data.

TABLE 4
Data collection breakdown by subject.

| General | | |
|---|---|---|
| Category | Behavior Type | Behavior Description |
| Inactive | Sleeping | No movement, not standing, eyes open |
| | Resting | No movement, not standing, eyes closed |
| Feeding | Free feeding | Head is in food bin, chewing |
| | Pedialyte | Pedialyte through cage doors while in-vivo |
| | | team exchanges batteries/fixes bandages: |
| | | head touching bottle |
| Drinking | Water Spigot | Head to water spigot |
| Playing | Hanging Toy Ball | Head or body touching the hanging ball |
| | Hanging gear | Head or body touching the gears |
| | Free moving toy | Interaction with head/body; moving |
| | Scratching/rooting | Pawing at or using their nose to move |
| | woodchips | woodchips |
| | Scratching or | Body up and touching the walls of the cage |
| | climbing | |
| | on walls of cage | |
| | Interaction with | Touching, nosing, being fed from |
| | researcher | researcher |

To quantify the feature detector's performance, five-fold cross validation was used across each hour of image data. The k-fold cross-validation included 1) division of the dataset into randomly sampled, independent k-folds without replacement, 2) K-1 folds used for model training and the remaining used for performance evaluation 3) repetition of prior step k times to obtain k number of performance estimates for each iteration and 4) a mean of k number of performance estimates. Then the performance of CRFs on test images across all generated features was evaluated.

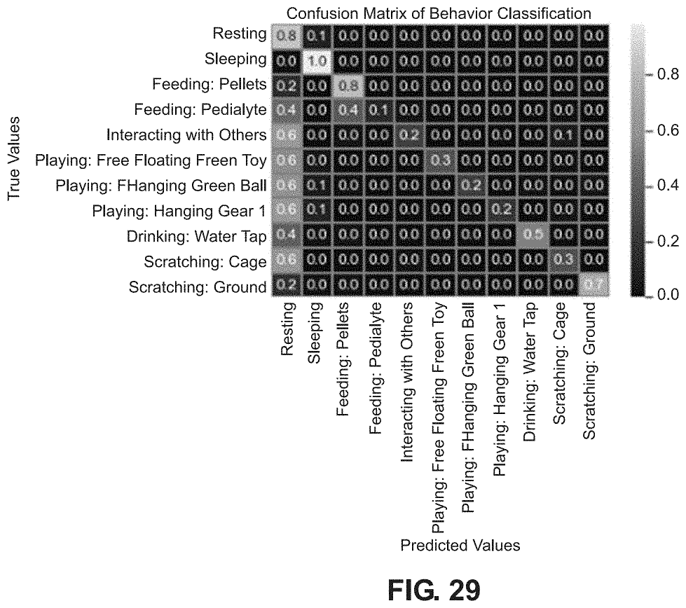

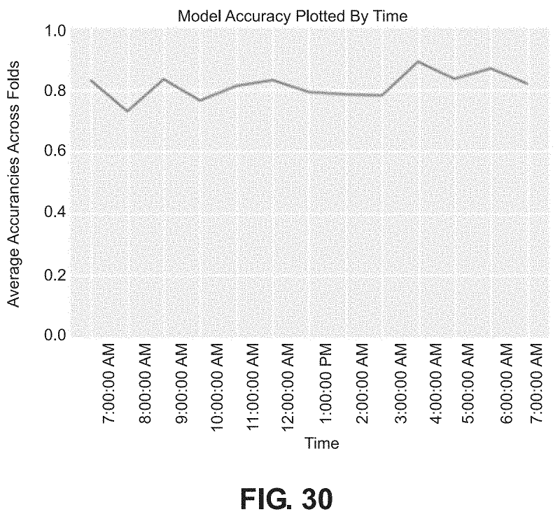

To capture model performance, the confusion matrix of the behaviors and the model's accuracy over time was provided. On average, the model classifies 11 different behavioral phenotypes with 81.1% accuracy for all 11 behaviors (FIGS. 29 and 30).

Histology

Following euthanasia, stomach sections were carefully harvested from animals in the control and experimental groups. Tissue samples were fixed in 4% paraformaldehyde for 24 hours. They were then washed in phosphate buffered saline three times for 15 minutes each and stored in 70% ethanol. They were then paraffin processed, embedded, and then sectioned. Tissues were stained with 1) hematotoxylin and eosin to assess morphology and surveil for adverse side effects related to the intervention.

Statistical Analyses

Quantitative data are reported as mean (±standard deviation) or as a range when appropriate. The normality of the distributions was checked by the Shapiro-Wilk test. Comparative analyses were performed using student's heteroscedastic two-tailed t-test, unless otherwise noted. P<0.05 was considered significant.

Additional Embodiments

In some embodiments, any ingestible capsule as described herein can further include one or more arms (e.g., a pair of arms) for gripping tissue such as, for example, gastric tissue that forms part of the stomach wall. For example, as described and illustrated in US Publication No. 2015/0051589, any ingestible capsule described herein can include a pair of arms that are disposed or retained within the form factor of the capsule (i.e., don't protrude) when not in use. Each arm can be coupled to a pin such that, when deployed, each arm can rotate about its respective pin to swing out and be substantially vertical to a longitudinal axis of the capsule. Each arm can be arranged to swing towards the other arm so as to pinch or grip tissue between them. In this manner, the ingestible capsule can be retained in the stomach for an extended period of time for medication delivery and treatment. As described in US Publication No. 2015/0051589, positioning and control of movement of the arms can be performed by a positioning circuit and a control circuit, respectively, either or both of which can be included in the electrical circuit described herein. The disclosure of US Publication No. 2015/0051589 is incorporated by reference in its entirety for all purposes.

In some embodiments, any ingestible capsule as described herein can further include one or more arms (e.g., a pair of arms) to modulate movement of the capsule through the GI tract. As an example, as described and illustrated in US Publication No. 2019/0209090, an ingestible capsule as described herein can include a pair of arms that are movable between a drawn-in configuration and a flared configuration where the arms splay out to increase the overall size of the capsule. As a result of this increased size, the capsule is prevented or hindered from exiting the stomach through the pyloric valve, such that the capsule can be retained in the stomach for an extended period of time for medication delivery and treatment. As another example and as also described in US Publication No. 2019/0209090, the arms can be retained and drawn back in when it is deemed that it is acceptable for the capsule to pass through the stomach. As another example and as also described in US Publication No. 2019/0209090, the arms can alternatively be formed such that they can mechanically separate from the body of the capsule due to degradation (e.g., due to gastric acid) of the coupling between the arms and the rest of the capsule. The coupling between the arms and the rest of the capsule can be designed so that the flared configuration is attained for some desired time period. As described in US Publication No. 2015/0051589, the deployment of the arms can be controlled via suitable electronic means, which can be included in the electrical circuit described herein. The disclosure of US Publication No. 2019/0209090 is incorporated by reference in its entirety for all purposes.

Conclusion

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of” “only one of,” or “exactly one of.” “Consisting essentially of” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An ingestible capsule comprising:
a housing forming a cavity;
a vibrator disposed in the cavity;
a power supply disposed in the cavity; and
a biodegradable insulating membrane disposed in electrical series with the vibrator and the power supply and in fluid communication with an exterior of the housing, the biodegradable insulating membrane configured to dissolve in a fluid having a pH of 1.5 to 9, thereby closing a circuit connecting the power supply and the vibrator.

2. The ingestible capsule of claim 1, wherein the vibrator comprises:
a motor having a shaft; and
a weight mechanically coupled to the shaft and radially offset from a longitudinal axis of the shaft.

3. The ingestible capsule of claim 2, wherein the shaft is configured to rotate about the longitudinal axis of the shaft at a frequency of about 20 Hz to about 300 Hz.

4. The ingestible capsule of claim 2, wherein, when the ingestible capsule is in a subject's stomach, the vibrator is configured to rotate the weight about the longitudinal axis of the shaft to generate a centrifugal force, thereby stroking a portion of mucosa in the subject's stomach with the ingestible capsule.

5. The ingestible capsule of claim 1, wherein the biodegradable insulating membrane comprises at least one of glucose, gelatin, ellastolan, cellulose, or Eudragit.

6. The ingestible capsule of claim 1, wherein the biodegradable insulating membrane is configured to dissolve in a fluid having a pH of 1.5 to 3.

7. The ingestible capsule of claim 1, further comprising:
a spring that is in a compressed state when in contact with the biodegradable insulating membrane and in an expanded state after the biodegradable insulating membrane has dissolved.

8. The ingestible capsule of claim 7, further comprising:
a pogo pin, wherein the spring is part of the pogo pin.

9. The ingestible capsule of claim 1, wherein:
the cavity is a first cavity; and
the housing comprises:
a first section;
a second section press-fittingly coupled with the first section to form the first cavity; and
a third section press-fittingly coupled with the second section to form a second cavity containing the biodegradable insulating membrane, the third section having a conduit for fluid communication between the second cavity and the exterior of the housing.

10. The ingestible capsule of claim 1, wherein the housing comprises a protruding member disposed on an outer surface of the housing.

11. The ingestible capsule of claim 10, wherein the protruding member has a helical or grooved shape.

12. The ingestible capsule of claim 10, wherein the protruding member is a plurality of studs protruding from an outer surface of the housing.

13. The ingestible capsule of claim 1, wherein, when the ingestible capsule is in a stomach of a subject who has ingested the ingestible capsule, closing the circuit connecting the power supply and the vibrator causes the vibrator to vibrate the ingestible capsule at a frequency that induces a feeling of satiety in the subject.

14. The ingestible capsule of claim 1, wherein, when the ingestible capsule is in a stomach of a subject who has ingested the ingestible capsule, closing the circuit connecting the power supply and the vibrator causes the vibrator to vibrate the ingestible capsule at a frequency that induces an illusory insufflation of the stomach.

15. The ingestible capsule of claim 1, wherein, when the ingestible capsule is in a stomach of a subject who has ingested the ingestible capsule, closing the circuit connecting the power supply and the vibrator causes the vibrator to vibrate the ingestible capsule at a frequency that induces serotonin release in the subject.

16. The ingestible capsule of claim 1, wherein, when the ingestible capsule is in a stomach of a subject who has ingested the ingestible capsule, closing the circuit connecting the power supply and the vibrator causes the ingestible capsule to stimulate mucosal receptors in the stomach.

17. An ingestible capsule comprising:
a housing forming a cavity;
an actuator disposed in the cavity and configured to oscillate about a longitudinal axis of the ingestible capsule at a frequency of about 5 Hz to about 120 Hz, thereby causing the ingestible capsule to rotate; and
a power supply disposed in the cavity and configured to provide power to the actuator.

18. The ingestible capsule of claim 17, wherein, when the ingestible capsule is in a subject's stomach, rotation of the ingestible capsule causes the ingestible capsule to stroke a portion of mucosa in the subject's stomach.

19. A method of stimulating a sensation of satiety in a subject, the method comprising:
with an ingestible capsule, stroking a portion of mucosa in a stomach of the subject at a frequency of about 50 Hz to about 120 Hz to induce an illusory distension of the stomach.

20. The method of claim 19, further comprising:
closing a circuit connecting a power supply and a vibrator in the ingestible capsule to induce the stroking by dissolving, with stomach fluid, a biodegradable insulating membrane disposed in electrical series between the power supply and the vibrator,
wherein the stroking the portion of mucosa comprises radially oscillating the ingestible capsule about a longitudinal axis of the ingestible capsule with the vibrator.

21. An ingestible capsule comprising:
a housing forming a cavity;
a vibrator disposed in the cavity;
a power supply disposed in the cavity;
a biodegradable insulating membrane disposed in electrical series with the vibrator and the power supply and in fluid communication with an exterior of the housing, the biodegradable insulating membrane configured to dissolve in stomach acid; and
a conductive spring held in a compressed state when in contact with the biodegradable insulating membrane and an expanded state closing a circuit between the vibrator and the power supply after the biodegradable insulating membrane is dissolved, thereby causing the vibrator to move about a longitudinal axis of the ingestible capsule at a frequency of about 5 Hz to about 120 Hz, motion of the vibrator causing the ingestible capsule to stroke a portion of mucosa when the ingestible capsule is in a subject's stomach.
